(19) United States
(12) Patent Application Publication
(10) Pub. No.: US 2025/0144311 A1
(43) Pub. Date: May 8, 2025

(54) NIPPLE SHIELD FLUSH SYRINGE AND STORAGE STERILIZING CASE

(71) Applicant: MUNCHKIN, INC., Van Nuys, CA (US)

(72) Inventors: Agnes Yena Lee, Los Angeles, CA (US); Sung Yun Chan, Pasadena, CA (US); Lucy Hui, Los Angeles, CA (US); Steven Bryan Dunn, Beverly Hills, CA (US)

(21) Appl. No.: 18/936,960

(22) Filed: Nov. 4, 2024

Related U.S. Application Data
(60) Provisional application No. 63/547,257, filed on Nov. 3, 2023.

(51) Int. Cl. A61M 5/315; A61J 13/00; A61L 2/07; B08B 9/032

(52) U.S. Cl. CPC A61M 5/31511; A61J 13/00; A61L 2/07; B08B 9/0321; A61L 2202/121; A61L 2202/122; A61L 2202/17; A61L 2202/24; B08B 2209/032; B08B 2230/01

(57) ABSTRACT

A flush syringe including a barrel, and a plunger. A tip of the barrel has a substantially flat surface that expands radially outward that then extends into an annular wall. The annular wall expands radially down and outward from the rounded edge to a tip base. The barrel defines a liquid reservoir and is configured to receive a plunger. The plunger is inserted into the barrel to forcibly dispense a fluid stored within the fluid reservoir through an outlet and into a mating device such as a nipple shield.

### CROSS REFERENCE TO RELATED APPLICATIONS

This application claims priority to U.S. Provisional Patent Application Ser. No. 63/547,257, filed Nov. 3, 2023, the content of which is hereby incorporated by reference herein in its entirety into this disclosure.

### TECHNICAL FIELD

The subject disclosure relates generally to syringes and breast feeding devices. More particularly, the present disclosure relates to a nipple shield flush syringe, a nipple shield sterilizing case and a storage container for the nipple shield flush syringe and the nipple shield.

### BACKGROUND

Often infant feeding devices such as a nipple shield encounter various fluids such as breastmilk, or saliva. These infant feeding devices such as a nipple shield, have small orifices and/or crevices where the various residual fluids can unintentionally be left behind. Typically, such devices are rinsed by hand with water or a disinfecting solution after use. If the devices are improperly cleaned, residue from the various fluids can remain within the orifices and/or crevices leading to bacterial build up, discoloration, or cause a foul odor rendering the device unsafe to use with an infant.

### SUMMARY

The following presents a simplified summary of the subject disclosure to provide a basic understanding of some aspects thereof. This summary is not an extensive overview of the various embodiments of the subject disclosure. It is intended to neither identify key or critical elements of the subject disclosure nor delineate any scope thereof. The sole purpose of the subject summary is to present some concepts in a simplified form as a prelude to the more detailed description that is presented hereinafter.

The present subject disclosure describes a flush syringe having a barrel, and a plunger. The barrel having a closed first end and an open second end. The closed first end is formed as a tip having an outlet. A wall of the barrel at the tip has a substantially flat surface that expands radially outward into a concentric barrel projection. The wall then flares slightly inward, and then extends outward and downward from the concentric barrel projection to a tip base. The wall continues to extend from the tip base uniform in diameter and cylindrically downward to the open second end. The barrel defines a liquid reservoir adapted to store a fluid that is bounded by the tip and the barrel. The plunger has a post and a stopper secured to the post. The stopper has a mating flat surface and a mating concentric projection adapted to fit within the interior of the substantially flat surface of the barrel and the concentric barrel projection. When the plunger is pushed into the open second end toward the tip of the barrel, the stopper is configured to sealingly engage the wall of the barrel and forcibly dispense the fluid stored in the fluid reservoir outward through the outlet. The flush syringe may be used with a mating device such as a nipple shield to rinse or sterilize the nipple shield.

The present subject disclosure further describes the use of the flush syringe with a sterilizing case. The sterilizing case has a receptacle having a perimeter wall surrounding a base. The base has a reservoir container into which a fluid may be placed. The base further has a projection extending upward a predetermined distance from within the reservoir container. The projection having at least one channel disposed therein through which the fluid may traverse upwardly. The sterilizing case further has a cover connected to the receptacle adapted to conceal the receptacle.

Furthermore, the present subject disclosure describes the use of the nipple shield and flush syringe with a sterilizing storage case. The sterilizing storage case has a receptacle having a perimeter wall surrounding a base. The base is divided into an upper base section and a lower base section. The upper base section encircles a reservoir container into which fluid may be placed. The upper base section further has a projection extending upward a predetermined distance from within the reservoir container. The projection having at least one channel disposed therein through which the fluid may traverse upwardly. The lower base forms a basin for which fluid may be placed. The lower base section further has a raised parabolic projection. The sterilizing storage case further has a cover connected to the receptacle adapted to conceal the receptacle.

### BRIEF DESCRIPTION OF THE DRAWINGS

Various exemplary embodiments of this disclosure will be described in detail, wherein like reference numerals refer to identical or similar components or steps. The accompanying drawings, which are incorporated in and constitute a part of this specification, illustrate embodiments of the subject disclosure and technical data supporting those embodiments, and together with the written description, explain certain principles of the subject disclosure. With reference to the following figures, wherein:

FIG. 1 is an upper perspective view of a flush syringe, according to an exemplary embodiment of the present subject disclosure.

FIG. 2 a cross-sectional front view of a barrel, according to an exemplary embodiment of the present subject disclosure.

FIG. 3 is a front view of the flush syringe, according to an exemplary embodiment of the present subject disclosure.

FIG. 4 is an exploded upper perspective view of the flush syringe, according to an exemplary embodiment of the present subject disclosure.

FIG. 5 is a front view of a stopper joined with a post, according to an exemplary embodiment of the present subject disclosure.

FIG. 6 is a cross sectional of the stopper joined with the post, according to an exemplary embodiment of the present subject disclosure.

FIG. 7 is a front view of the stopper, according to an exemplary embodiment of the present subject disclosure.

FIG. 8 is an upper perspective view of the stopper, according to an exemplary embodiment of the present subject disclosure.

FIG. 9 is a cross section view of the stopper joined with the post and inserted into the barrel, according to an exemplary embodiment of the present subject disclosure.

FIG. 10 is a cross section view of the stopper, according to an exemplary embodiment of the present subject disclosure.

FIG. 11A is a lower perspective view of the stopper, according to an exemplary embodiment of the present subject disclosure.

FIG. 11B is a bottom view of the stopper, according to an exemplary embodiment of the present subject disclosure.

FIG. 12 is a front view of the post, according to an exemplary embodiment of the present subject disclosure.

FIG. 13 is a top view of the flush syringe, according to an exemplary embodiment of the present subject disclosure.

FIG. 14 is a bottom view of the flush syringe, according to an exemplary embodiment of the present subject disclosure.

FIG. 15 is a cross sectional view of the flush syringe in use with a basin, drawing in a fluid into a fluid reservoir, according to an exemplary embodiment of the present subject disclosure.

FIG. 16 is a front view of the flush syringe in use with a nipple shield, according to an exemplary embodiment of the present subject disclosure.

FIG. 17A is a cross sectional view of the flush syringe in use with the nipple shield illustrating the insertion of the fluid through a channel in the nipple shield to sterilize the nipple shield, according to an exemplary embodiment of the present subject disclosure.

FIG. 17B shows an enlarged portion A-A of the cross sectional view of the flush syringe shown in FIG. 17A in use with the nipple shield illustrating the insertion of the fluid through a channel in the nipple shield to sterilize the nipple shield, according to an exemplary embodiment of the present subject disclosure.

FIG. 18 is a front view of the flush syringe dispensing the rinse fluid through the nipple shield, according to an exemplary embodiment of the present subject disclosure.

FIG. 19 is an upper perspective of the flush syringe dispensing the fluid through the nipple shield, according to an exemplary embodiment of the present subject disclosure.

FIG. 20 is a right side view of the flush syringe dispensing the fluid through the nipple shield, according to an exemplary embodiment of the present subject disclosure.

FIG. 21A is a top view of the nipple shield, according to an exemplary embodiment of the present subject disclosure.

FIG. 21B is an upper perspective view of the nipple shield in use with an infant, according to an exemplary embodiment of the present subject disclosure.

FIG. 22 is an upper perspective view of a sterilizing case in a closed position, according to an exemplary embodiment of the present disclosure.

FIG. 23 is a top view of the sterilizing case in a closed position, according to an exemplary embodiment of the present subject disclosure.

FIG. 24 is a front view of the sterilizing case in a closed position, according to an exemplary embodiment of the present subject disclosure.

FIG. 25 is a back view of the sterilizing case in a closed position, according to an exemplary embodiment of the present subject disclosure.

FIG. 26 is a top view of the sterilizing case in an open position, according to an exemplary embodiment of the present subject disclosure.

FIG. 27A is an upper perspective view of the sterilizing case in an open position, according to an exemplary embodiment of the present subject disclosure.

FIG. 27B is an upper perspective left side view of the sterilizing case in an open position, according to an exemplary embodiment of the present subject disclosure.

FIG. 28 is a right side view of the sterilizing case in a closed position, according to an exemplary embodiment of the present subject disclosure.

FIG. 29 is a bottom perspective view of the sterilizing case in a closed position, according to an exemplary embodiment of the present subject disclosure.

FIG. 30 is a bottom view of the sterilizing case in a closed position, according to an exemplary embodiment of the present subject disclosure.

FIG. 31 is an upper perspective view of the sterilizing case in an open position in use with the nipple shield, according to an exemplary embodiment of the present subject disclosure.

FIG. 32 is a top view of the sterilizing case in an open position in use with the nipple shield, according to an exemplary embodiment of the present subject disclosure.

FIG. 33 is a cross sectional view of the sterilizing case in a closed position while sterilizing the nipple shield, according to an exemplary embodiment of the present subject disclosure.

FIG. 34 is an upper perspective view of a sterilizing case in a close position, according to an alternative exemplary embodiment of the present subject disclosure.

FIG. 35 is a top view of the sterilizing case in a closed position, according to an exemplary embodiment of the present subject disclosure

FIG. 36 is a front view of the sterilizing case in a closed position, according to an exemplary embodiment of the present subject disclosure.

FIG. 37 is a bottom perspective view of the sterilizing case in a closed position, according to an exemplary embodiment of the present subject disclosure.

FIG. 38 is a bottom view of the sterilizing case in a closed position, according to an exemplary embodiment of the present subject disclosure.

FIG. 39 is an upper perspective view of the sterilizing case in an open position, according to an exemplary embodiment of the present subject disclosure.

FIG. 40 is a cross sectional view of the sterilizing case in a closed position, according to an exemplary embodiment of the present subject disclosure.

FIG. 41 is a top view of the sterilizing case in an open position, according to an exemplary embodiment of the present subject disclosure.

FIG. 42 is a top view of the sterilizing case in an open position in use with the nipple shield, according to an exemplary embodiment of the present subject disclosure.

FIG. 43 is an upper perspective view of the sterilizing case in an open position in use with the nipple shield, according to an exemplary embodiment of the present subject disclosure.

FIG. 44 is an upper perspective view of a sterilizing storage case, according to an exemplary embodiment of the present subject disclosure.

FIG. 45 is a top view of the sterilizing storage case in a closed position, according to an exemplary embodiment of the present subject disclosure.

FIG. 46 is a back view of the sterilizing storage case in a closed position, according to an exemplary embodiment of the present subject disclosure.

FIG. 47 is a front view of the sterilizing storage case in a closed position, according to an exemplary embodiment of the present subject disclosure.

FIG. 48 is a right view of the sterilizing storage case in a closed position, according to an exemplary embodiment of the present subject disclosure.

FIG. 49 is a bottom view of the sterilizing storage case in a closed position, according to an exemplary embodiment of the present subject disclosure.

FIG. 50 is a top view of the sterilizing storage case in an open position, according to an exemplary embodiment of the present subject disclosure.

FIG. 51 is an upper perspective view of the sterilizing storage case in an open position, according to an exemplary embodiment of the present subject disclosure.

FIG. 52 is a right side view of the sterilizing storage case in an open position, according to an exemplary embodiment of the present subject disclosure.

FIG. 53 is a front view of the sterilizing storage case in an open position, according to an exemplary embodiment of the present subject disclosure.

FIG. 54 is an upper perspective view of the sterilizing storage case in an open position in use with the nipple shield and flush syringe, according to an exemplary embodiment of the present subject disclosure.

FIG. 55 is a top view of the sterilizing storage case in an open position in use with the nipple shield and flush syringe, according to an exemplary embodiment of the present subject disclosure.

FIG. 56 is a side view of the sterilizing storage case in an open position in use with the nipple shield and flush syringe, according to an exemplary embodiment of the present subject disclosure.

FIG. 57 is a flow diagram for the sterilizing storage case method of use, according to an exemplary embodiment of the present subject disclosure.

FIG. 58 is a top view of the sterilizing storage case in an open position in an alternate use with the nipple shield and flush syringe, according to an exemplary embodiment of the present subject disclosure.

FIG. 59 is a cross sectional view of the sterilizing storage case in an open position in use with the nipple shield and flush syringe, according to an exemplary embodiment of the present subject disclosure.

FIG. 60 is a cross sectional view of the sterilizing storage case in a closed position sterilizing the nipple shield and flush syringe, according to an exemplary embodiment of the present subject disclosure.

FIG. 61 is an upper perspective view of a sterilizing storage case in a closed position, according to an alternative exemplary embodiment of the present subject disclosure.

FIG. 62 is an upper perspective view of a sterilizing storage case in an open position, according to an alternative exemplary embodiment of the present subject disclosure.

FIG. 63 is an upper perspective view of a sterilizing storage case in an open position in use with the nipple shield and flush syringe, according to an alternative exemplary embodiment of the present subject disclosure.

FIG. 64 is a top view of a sterilizing storage case in an open position in use with the nipple shield and flush syringe, according to an alternative exemplary embodiment of the present subject disclosure.

FIG. 65 is a side view of a sterilizing storage case in an open position in use with the nipple shield and flush syringe, according to an alternative exemplary embodiment of the present subject disclosure.

FIG. 66 is a front view of a sterilizing storage case in an open position with a heating plate in use with the nipple shield and flush syringe, according to an alternative exemplary embodiment of the present subject disclosure.

### DETAILED DESCRIPTION

The subject disclosure is described with reference to the drawings, wherein like reference numerals are used to refer to like elements throughout. In the following description, for purposes of explanation, numerous specific details are set forth to provide a thorough understanding of the present disclosure. It may be evident, however, that the present disclosure may be practiced without these specific details. An exemplary embodiment of a flush syringe will now be described in greater detail with reference to the figures.

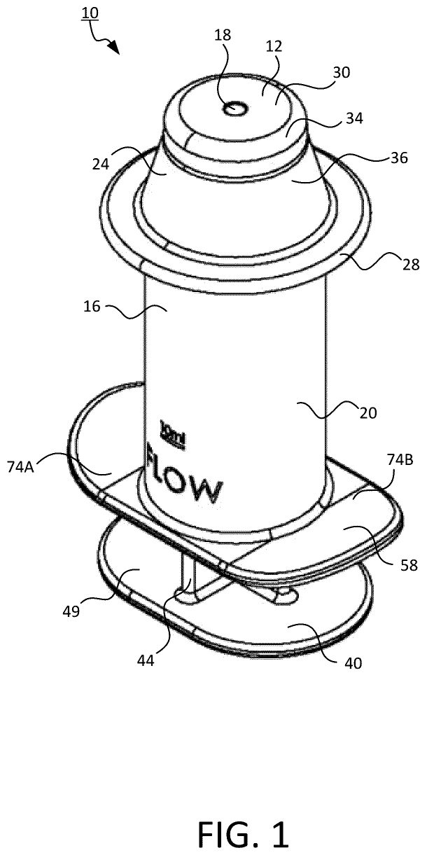

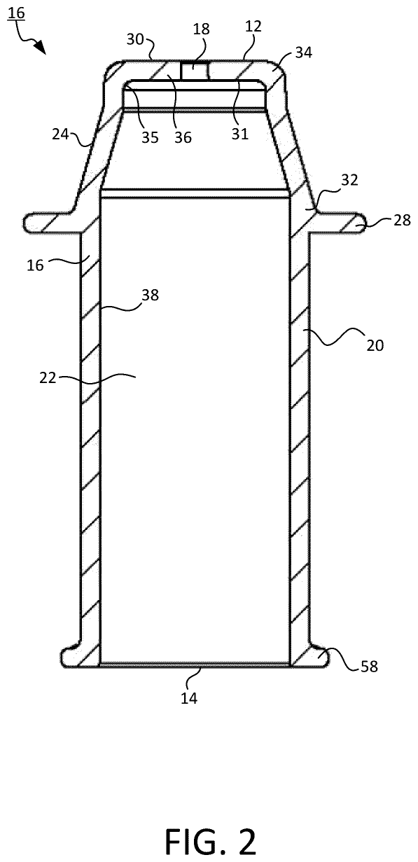

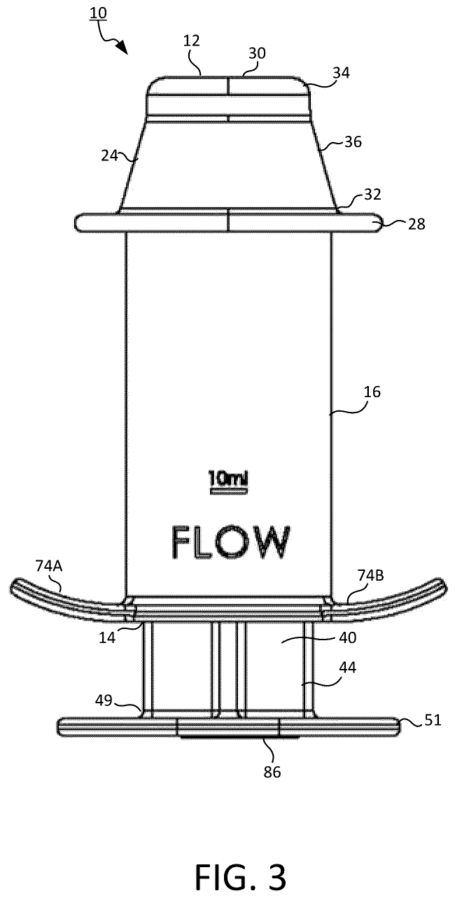

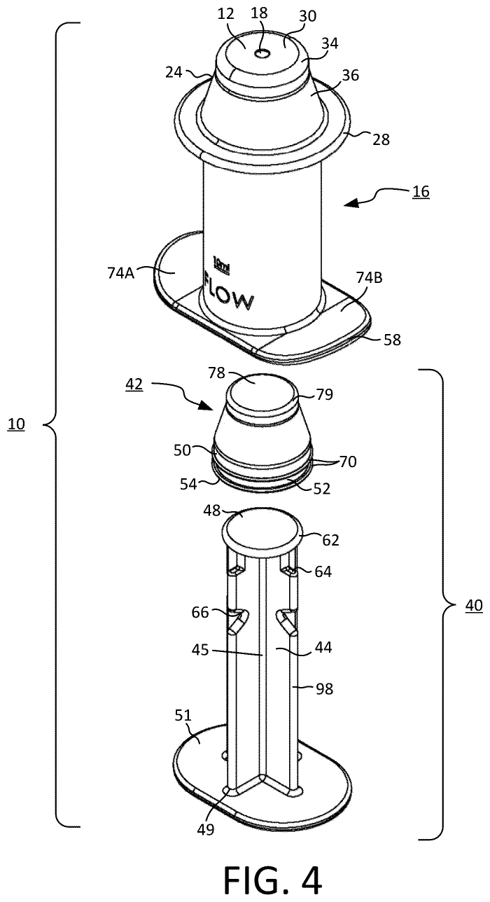

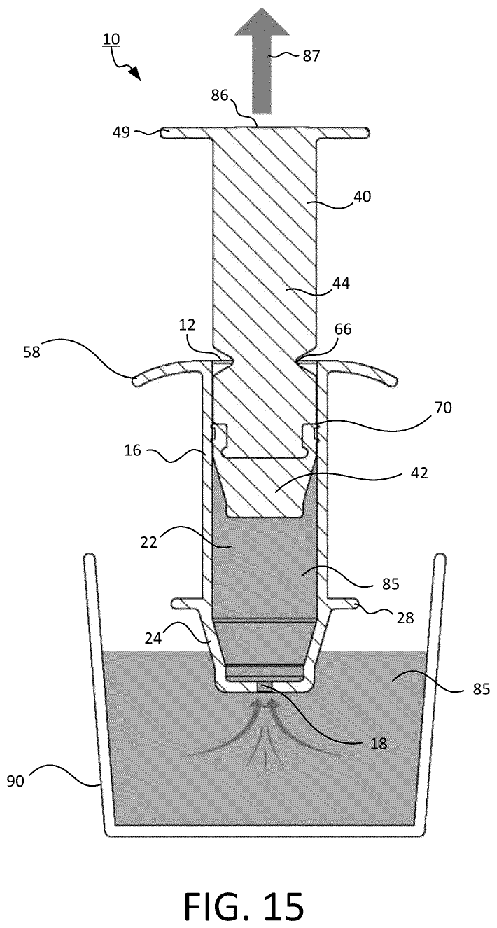

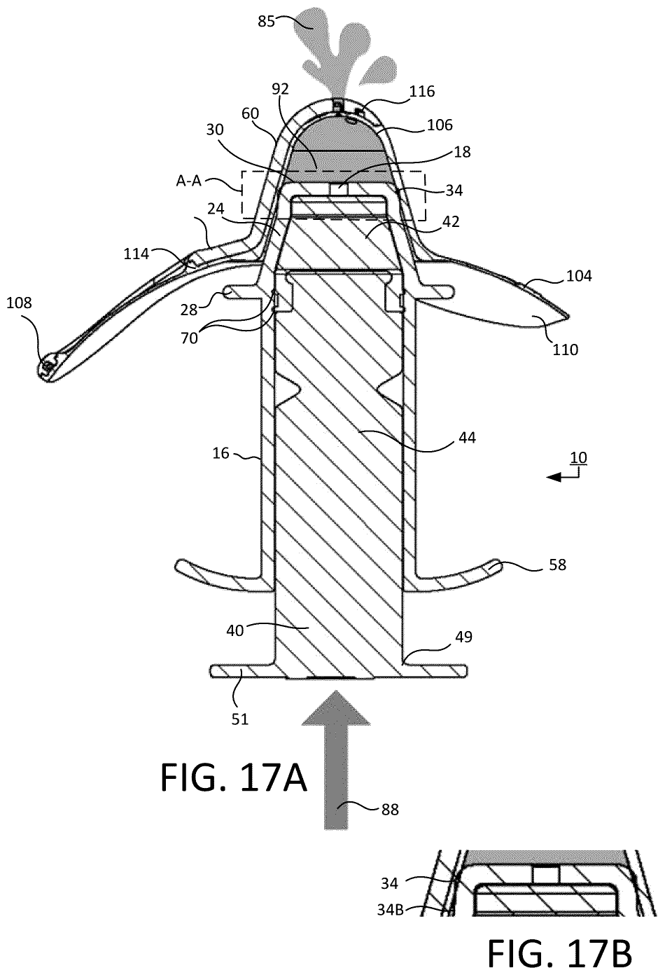

FIGS. 1-4 illustrate a flush syringe 10, having a barrel 16, and a plunger 40. The barrel 16 has a closed first end 12 and an open second end 14, where the closed first end 12 forms a tip 24 having an outlet 18. A wall 36 of the barrel 16 at the tip 24 has a substantially flat surface 30 that expands radially outward into a concentric barrel projection 34 which acts as an outwardly projected lip, has a rounded edge and flares outwardly from the top surface. The wall 36 then flares slightly inward from the concentric barrel projection 34, and then extends downward and outward from the concentric barrel projection 34 to a tip base 32. See FIG. 17A-17B for more detailed view of the outwardly projected lip 34. The wall 36 then extends from the tip base 32 uniform in diameter and cylindrically downward to the open second end 14. The barrel 16 is a single uniform piece, comprised of a wall substantially uniform in size and thickness, and defines a fluid reservoir 22 adapted to store a fluid 85 (FIG. 15) that is bounded by the tip 24, the barrel 16, and the plunger 40.

It is to be understood that the tip 24 may have alternative embodiments with varying shapes such as, but not limited to: frustoconical, dome, triangular, square, or equivalent shape to meet the function described by the flush syringe 10 of this present disclosure. For example, the tip 24 may be sealingly fitted to a mating device that requires a triangular shape to ensure a proper fit and seal.

Furthermore, it is to be understood that the outlet 18 may be centered or displaced within the tip 24 and includes at least one aperture suitably capable of dispensing the fluid 85 through the tip 24 of the barrel 16 as shown and described by the flush syringe 10 of this subject disclosure. For example, the tip 24 may have a plurality of the outlets 18 disposed about the substantially flat surface 30 or a singular outlet 18 that is off-center with the tip 24 to control the volumetric flow of the fluid 85 released from fluid reservoir through the outlet 18.

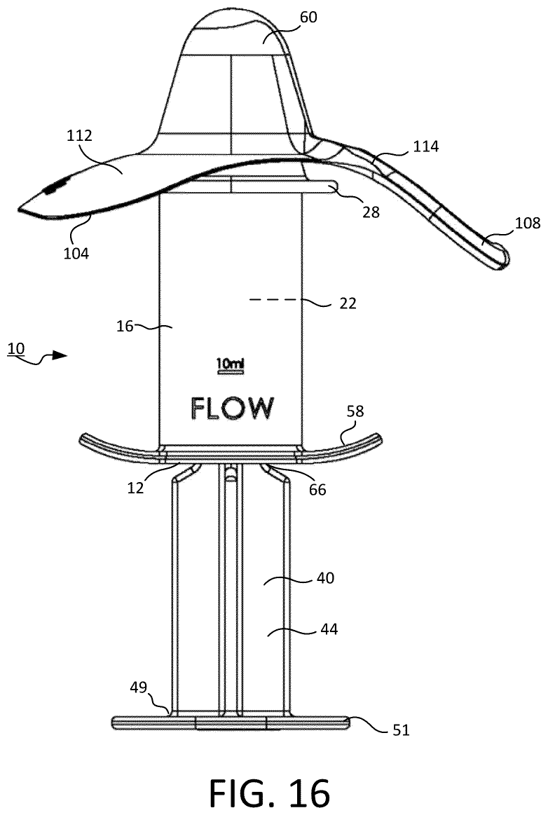

The flush syringe 10 may have a splash guard 28, that extends radially outward from the tip base 32 by a predetermined length. The splash guard 28 may serve as a leverage flange for a user to grip onto when inserting the tip 24 into a mating device, such as a nipple shield 60, see FIGS. 16 and 17. For example, the user may utilize the splash guard 28 to anchor their fore fingers and depress the nipple shield 60 with their thumb or other hand. The splash guard 28 may also trap or deflect escaping fluid 85 when the tip 24 is mated with a mating device when an improper seal is formed, deflecting the fluid 85 away from the user's hand. Further, the splash guard 28 may serve as a visual indicator to tell the user how far the tip 24 is to be inserted into a mating device. For example, when inserting the flush syringe 10 to a mating device, such as a nipple shield 60, the user will place the nipple shield 60 over the tip 24 and depress the nipple shield 60 over the tip 24 to align the base of the nipple shield 60 with the splash guard 28. The user could visually utilize the decreasing distance between the splash guard 28 and the nipple shield 60 until a maximum insertion of the tip 24 is reached. In acting as a visual indicator, the splash guard 28 ensures a secure seal is made between the tip 24 and the mating device.

As shown best in FIG. 17A and the portion A-A shown in enlarged detail in FIG. 17B, a seal is established between the tip 24 and the mating device, such as a nipple shield 60, due to the slight deformation of the mating device caused by pressure fitment with the concentric barrel projection 34. The extended rounded edge 34 acts as a lip with a larger diameter than the surface 34B immediately below the rounded lip portion 34. Thus, the concentric barrel projection 34 is configured with a slightly larger diameter than the inner diameter of the mating device such that when the concentric barrel projection 34 is inserted into the mating device, the surface of the mating device is slightly deformed, thus forming a seal. It is to be understood that material of the extended rounded lip edge 34 is of an elastic type, such that temporary, slight deformation may occur such as but not limited to: silicone, rubber, or any other alternative elastic material capable of performing the described features as in this subject disclosure. In having the mating device be composed of an elastic material, it ensures the proper seal between the tip 24 and the mating device. For example, If the user places the mating device atop the tip 24 in a non-perpendicular alignment with tip 24, a seal still may be formed between an outer surface of the concentric barrel projection 34 and the surface of the mating device because the slight deformation may still occur due to the configured diameters thereby sealing the connection.

The fluid reservoir 22 is enclosed by the wall 36 having a continuous, uniform and predetermined thickness suitably capable of withstanding the dispensing pressure caused by the plunger 40 being depressed within the barrel 16 when rinsing or cleaning a mating device as described in this subject disclosure. The interior of the wall 36 extends from the open second end 14 uniform in diameter and cylindrically upward to the tip base 32. The interior of the wall 36 then narrows inward and upward to an inner concentric barrel projection 35. The interior of the wall 36 then radially contracts inward from the inner concentric barrel projection 35 to an inner substantially flat surface 31 in order to mate with the stopper 42 as described in more detail below. The outlet 18 is bored through the closed first end 12 to connect the fluid reservoir 22 with the atmosphere, permitting the fluid 85 to forcibly exit the fluid reservoir 22 when the plunger 40 is depressed into the barrel 16. See FIGS. 16-17.

The fluid reservoir 22 may vary in volumetric capacity such as, but not limited to: 10 milliliters, 20 milliliters or any volumetric measurement suitably capable of holding the fluid 85 and performing the features and attributes described such as rinsing, cleaning, flushing a mating device, such as the nipple shield 60, as described in this subject disclosure.

The material characteristics of the barrel 16 may vary to include, but not limited to: silicone, plastic, glass, metal, rubber or any suitable material capable of withstanding the dispensing pressure when the plunger 40 is depressed into the barrel 16 to flush, rinse or clean a mating device as described in this subject disclosure.

It is to be understood that the barrel 16, generally, is of one singular piece; however, in alternative embodiments the barrel 16 may comprise of multiple pieces joined together. For example, in an alternative embodiment, the tip 24 may be joined with a cylindrical container to form the barrel 16. The multiple pieces may be joined through one of many different methods which may include, but not limited to: adhesion, friction fitting, fastening or a suitable method capable of adhering the multiple pieces such that when combined they are capable of withstanding the dispensing pressure when the flush syringe 10 is in use with the plunger 40 to flush, rinse or clean a mating device as described in this subject disclosure. Permitting the barrel 16 to be made of multiples pieces may allow the tip 24 to have different material characteristics than the cylindrical container 20.

A different material characteristic for the tip 24 may provide for better fitment with a mating device such as the nipple shield 60 but the cylindrical container may be constructed of a different material. For example, if the mating device, such as the nipple shield 60, is made of silicone, the tip 24 made of glass may provide for a better seal but the cylindrical container made of plastic and may have increased performance capable of better withstanding the related pressure forces of rinsing, flushing or cleaning the mating device. Further, the barrel 16 made of multiples pieces permits the tip 24 to be interchangeable with alternate embodiments and/or for a secondary use. For example, the tip 24 may in one instance be frustoconical in shape to mate with the nipple shield 60. In a second instance, the tip 24 can be interchanged with an alternate embodiment for secondary use with a different mating device, such as rinsing a baby bottle nipple.

The barrel 16 may have a barrel flange 58. The barrel flange 58 extends bilaterally outward a predetermined length from the open second end 14. The barrel flange 58 may flare upward at a predetermined angle to create a recess 74A, 74B. For example, a user may anchor or rest their index finger and middle finger on the recess 74A, 74B respectively to leverage the barrel flange 58 against the plunger 40 when holding the flush syringe 10.

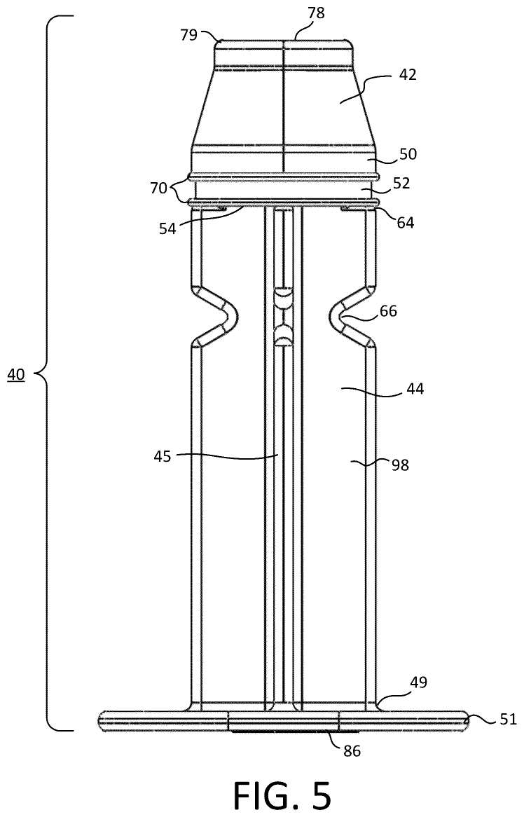

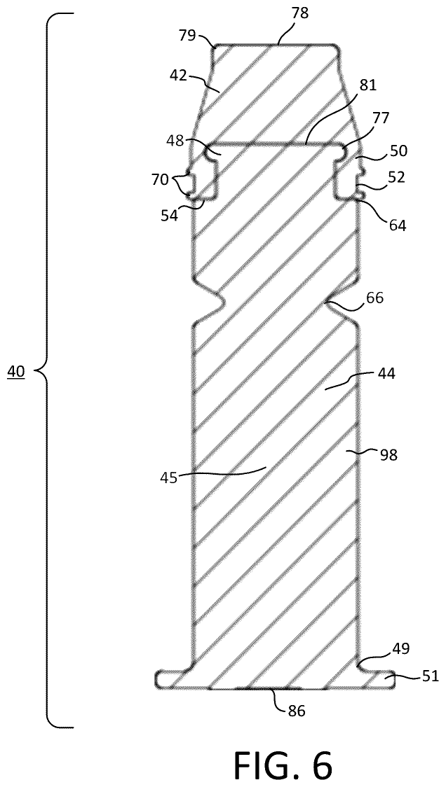

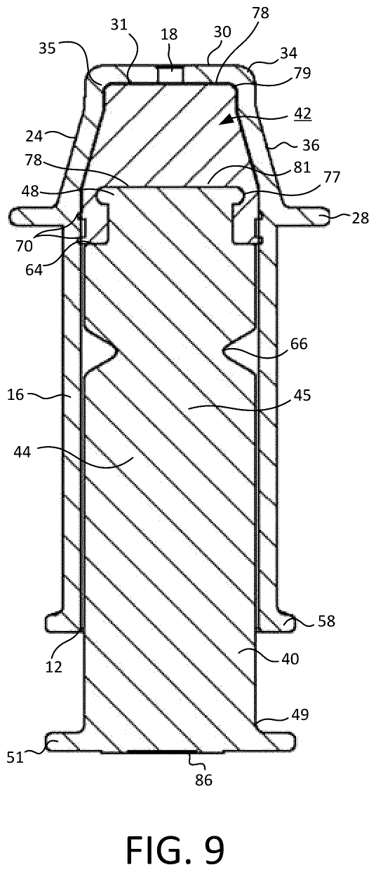

FIGS. 5 and 6 show the plunger 40 having a post 44 and a stopper 42 secured to the post 44. The stopper 42 having a flat surface 78 and a concentric stopper projection 79 adapted to mate with the inner substantially flat surface 31 and the inner concentric barrel projection 34 of the barrel 16. As shown in FIG. 9, when the plunger 40 is pushed into the open second end 14 of the barrel 16, toward the tip 24, the stopper 42 sealingly engages the wall 36 and forcibly dispenses the fluid 85 through the outlet 18, see also FIG. 17.

FIGS. 5-12 show the stopper 42 may be removably secured to the post 44. The stopper 42 may be attached using many different securing methods such as, but not limited to: fastening, friction fitting, adhesion or equivalent securing method capable of withstanding the pressure forces relating to the application of the flush syringe 10 as described by the subject disclosure. In permitting the stopper 42 to be removably securable to the post 44, the stopper 42 can be interchangeable for ease of replacement. For example, the stopper 42 may no longer create a proper seal with the wall 36 of the barrel 16 due to long term use but the post 44 may still be in good condition. In this instance, the stopper 42 may be interchanged with a new one to allow for continued use of the flush syringe 10. Further, the stopper 42 may vary in shape such as, but not limited to a: dome, frustoconical, square or any suitable shape capable of creating a proper seal with the wall 36 of the barrel 16 as described by this subject disclosure. It is to be understood that the stopper 42 may in alternate embodiments be permanently secured to the post 44 through one of many different joining methods such as, but not limited to: molding, die casting, adhesion, or any suitable method capable of sealing the stopper 42 to the post 44.

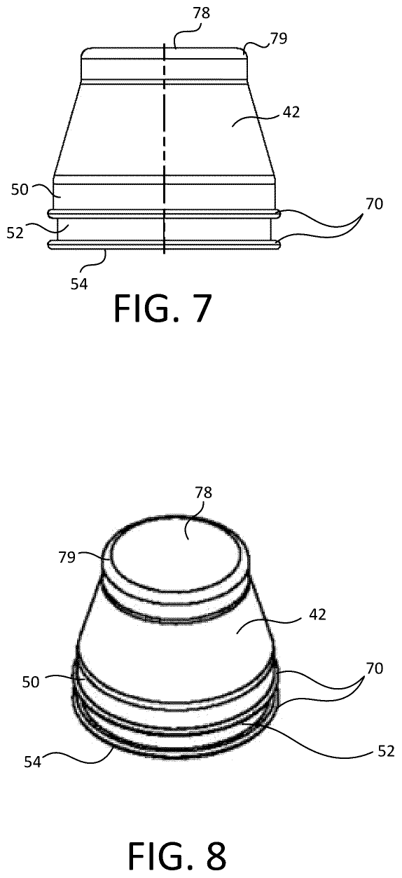

FIGS. 7-10 illustrate the stopper 42 having the flat surface 78 expand radially outward into the concentric stopper projection 79. The stopper 42 then flares slightly inward from the concentric stopper projection 79, and then extends outward and downward from the concentric stopper projection 79 to a stopper midsection 50. This configuration is similar to and substantially parallels the outer configuration of the top of the closed end 12 and lip projection 34. The stopper 42 then extends from the stopper midsection 50 uniform in diameter and cylindrically downward to a stopper lower section 52. The stopper 42 then radially contracts inward from stopper lower section 52 and then extends uniform in diameter and cylindrically downward to a stopper base 54.

The stopper 42 has a plurality of annular bulges 70, disposed between the stopper midsection 50 and at the stop base 54. In the present embodiment, at least one of the plurality of annular bulges 70 is located at the stopper lower section 52, and at least one of the plurality of annular bulges 70 is located at the stopper base 54.

As illustrated in FIG. 9, the plunger 40 is configured to fit within the barrel 16, such that the post 44 does not come into contact with the interior of the wall 36. The plurality of annular bulges 70 on the stopper 42 are configured to have a diameter slightly greater than the diameter than the stopper midsection 50 and the stopper lower section 52 of the stopper 42. The plurality of annular bulges 70 is also slightly greater in diameter than the inner diameter of the wall 36 that forms the fluid reservoir 22, such that the plurality of annular bulges 70 deform slightly to then form a seal with the interior of the wall 36 of the barrel 16. For example, when the plunger 40 is inserted into the barrel 16, the plurality of annular bulges 70 form a seal with and the wall 36 due to the difference in diameter to keep the fluid 85 (see FIG. 17) from escaping the fluid reservoir 22 backwards through the closed first end 12 of the barrel 16. The plurality of annular bulges 70 having a slightly greater diameter than the inner diameter of the wall 36, and the annular bulges 70 can flex inwardly and/or outwardly as they contact the wall 36. Accordingly, the static friction force formed between the plurality of annular bulges 70 and the wall 36 are reduced by the inward/outward flexation of the plurality of bulges 70 permitting the movement of the plunger 40 through the barrel 16 without breaking the seal formed between the plurality of annular bulges 70 and the wall 36. Further, the seal formed between the plurality of annular bulges 70 and the wall 36 may act as a guide for the plunger 40 when inserted into the barrel 16. For instance, when the plunger 40 is inserted into the barrel 16, the size of the post 44 is of a lesser diameter than the wall 36 to permit movement of the plunger 40, however, if the spatial gap formed by the difference in diameters is too large, there may tendency for the post 44 to move in the horizontal direction. As such, the plurality of annular bulges 70 may serve as a guide to restrict movement in the horizontal direction due to the seal formed with the wall 36 and encourages movement in the vertical, unilateral direction.

The material characteristics that may be considered for the stopper 42 and plurality of annular bulges 70 may vary to include, but are not limited to: silicone, rubber, or any suitable flexible material capable of withstanding the pressure forces of a depressed plunger 40 within the barrel 16 and permit the slight deformation of the plurality of annular bulges 70 as described in this subject disclosure.

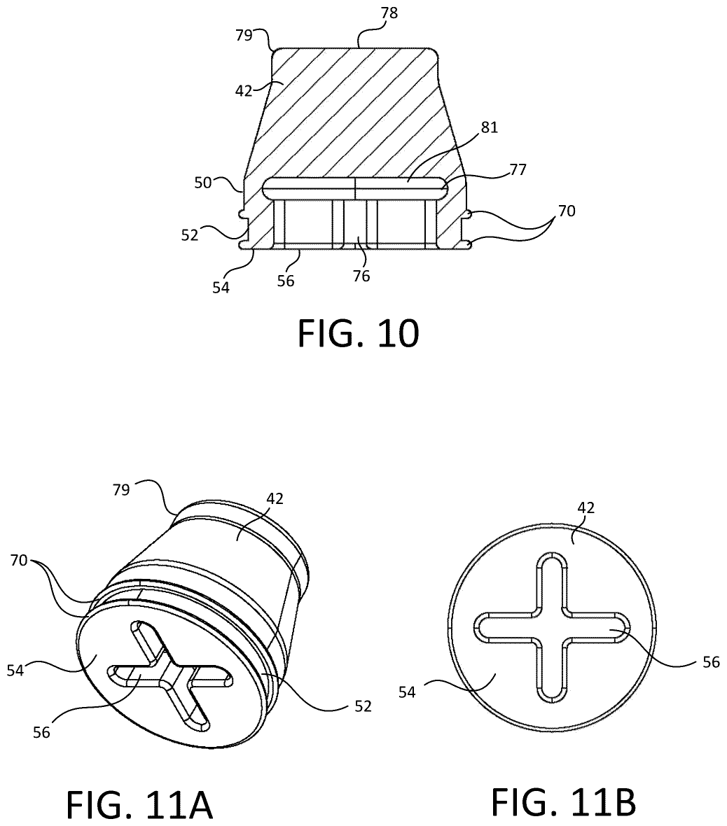

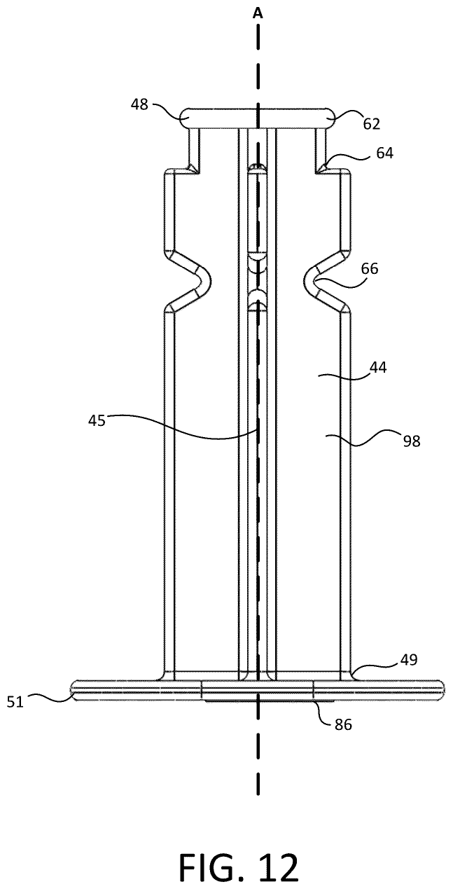

FIG. 10 illustrates an exemplary construction for the stopper 42. A blind face bore 76 maybe centered within the stopper 42. The blind face bore 76 extends upwardly from the stopper base 54 and then laterally outward into an inner rounded edge 77. The blind face bore 76 then extends radially inward from the inner rounded edge 77 having a stopper substantially flat surface 81. The blind face bore 76 is configured to receive a post tip 48 of the post body 45, see FIGS. 9 and 12.

As shown in FIG. 11A-B, the stopper base 54 has an aperture 56 centrally located, to allow the post tip 48 to be removably secured into the stopper 42. For example, the post tip 48 can be inserted through the aperture 56 and press fit to mate within the blind face bore 76. The aperture 56 is generally in the shape that conforms with the post ribs 98 so that the post tip 48 can be received within the blind face bore 76. The aperture 56 may take various shapes, as but not limited to a cross shape or any shape capable of suitably securing the post tip 48 into the blind face bore 76 as described by the flush syringe 10 of this subject disclosure. The blind face bore 76 is configured to receive post tip 48 such that the stopper base 54 is in communication with a locking indent 64 of the post 40, see FIG. 5. The post tip 48 is securely disposed between the blind face bore 76 and the locking indent 64 of FIG. 12. The locking indent 64 ensures that the stopper 42 does not separate from the post 44 keeping the post tip 48 in compression with the blind face bore 76.

FIG. 12 depicts the post 44 having the post tip 48 that expands radially outward to a rounded edge 62. In cross-section, lateral to an axial length A, of the post 44, the construction of the post 44 is a cross shape, with each of the post ribs 98 being a leg of the cross shape. The post tip 48 is attached to the post ribs 98 at one end. It is to be understood that the post 44 may have alternative embodiments, such as but not limited to, a solid cylindrical core or any suitable shape capable of performing the features and attributes described by the flush syringe 10 of this subject disclosure. The post 44 then extends axially downward from the rounded edge 62 to the locking indent 64. The post ribs 98 then extend outward and axially downward from the locking indent 64 to a post base 49.

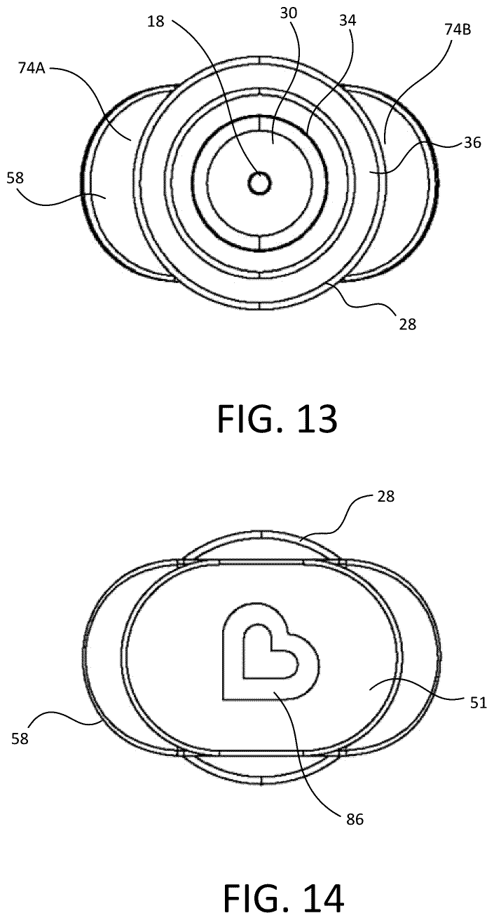

The post base 49 may extend bilaterally outward to form a base flange 51, see also FIGS. 1, 3 and 4. The base flange 51 may be utilized as leverage to a user when depressing the plunger 40 into the barrel 16. For example, the base flange 51 may permit more surface area for a user to utilize when depressing the plunger 40 with their thumb. As shown in FIGS. 13 and 14, the barrel flange 58 may extend bilaterally outward farther than the base flange 51 for added user comfort when gripping the flush syringe 10. For example, when the user grips the flush syringe 10 by placing their fore fingers in the recess 74A-B, and places their thumb upon the base flange 51, a shorter base flange 51 may encourage more comfort when held than a larger base flange 51. It is to be understood that the base flange 58 may have alternative embodiments such as, but not limited to, extending outwardly: the same distance, a greater distance or any other length suitably capable of providing leverage to a user when applying a compressive force to the plunger 40 as presented in this subject disclosure. The post base 49 further may have a protrusion 86 to serve as an additional grip for the user's thumb. For example, the protrusion 86 may prevent the user's thumb from slipping off the post base 49 when compressing the plunger 40 into the barrel 16.

Various materials that may be considered for the post 44 may including, but is not limited to: silicone, plastic, glass, metal, rubber, synthetic material and/or any suitable material capable of performing the features and attributes described by the flush syringe 10 of this subject disclosure.

Referring to FIG. 16 and FIG. 12, a full line indicator 66 is equally and radially disposed on the post ribs 98 at a predetermined location positioned between the locking indent 64 and the post base 49. The location of the full line indictor 66 is dependent on the predetermined volumetric capacity of the fluid reservoir 22. For example, if the volumetric capacity of the fluid reservoir is 10 or 20 milliliters, the full line indicator 66 informs a user of the maximum amount of the fluid 85 that the fluid reservoir 22 with the placement of the full line indicatory 66 higher or lower respectively. The full line indicator 66 is defined by a concave cutout. The full line indicator 66 may have alternative configurations such as, but limited to: rectangular cutout, triangular cutout, a line printed indicator or any other configurations that may serve as a visual indicator capable of performing the features presented in this subject disclosure. As shown in FIG. 16, the full line indicator 66 is configured such that when the plunger is extracted from the barrel 16, the mid-plane of the full line indicator 66 is coplanar with the open second end 14 indicating the predetermined volumetric capacity within the fluid reservoir 22. For example, when the midplane of full line indicator 66 is coplanar with the open second end 14 the fluid reservoir 22 has met a volumetric capacity.

FIGS. 15-18 demonstrate the flush syringe 10 in use. FIG. 15 depicts a barrel filling step (“BFS”), and FIG. 17A illustrates a fluid dispensing step (“FDS”). In the BFS, the plunger 40 is extracted outward from the barrel 16 by a first external force 87. In the FDS, the plunger 40 is depressed into the barrel 16 by a second external force 88 such that the stopper substantially flat surface 81 of the stopper 42 is mated with the inner substantially flat surface 31 of the barrel 16.

FIG. 15 depicts the flush syringe 10 in the BFS, receiving the fluid 85 to be contained within the fluid reservoir 22. The flush syringe 10 receives the fluid 85 by placing the tip 24 in the fluid 85, the fluid 85 as depicted may be withdrawn from a basin 90. By applying the first external force 87 to the plunger 40 while the tip 24 remains within the fluid 85. In applying the first external force 87 to the plunger 40, a negative pressure within the fluid reservoir 22 forms causing the fluid 85 to enter the fluid reservoir 22. As the plunger 40 is pulled away from the barrel 16 by the first external force 87, the voided space within the fluid reservoir 22 is filled with the fluid 85. It is to be understood that the flush syringe 10, is not limited for use with the basin 90 to engage with the fluid 85. The flush syringe 10, may be, but is not limited to usage with: containers, faucets or other suitable liquid sources for housing the fluid 85 as described by this subject disclosure.

FIG. 16 illustrates the flush syringe 10 filled by the BFS and mated with the nipple shield 60. It is to be understood that the flush syringe 10 may be used with varying mating devices and is not limited to the nipple shield 60. In alternative embodiments, the flush syringe 10 may be used, but not limited, to: feed an infant baby with a nutritional liquid source, flush breast pump hose lines, flush portable breast pump parts, flush baby bottles, flush baby bottle nipples, flush straws, flush no-spill cup components, flush nose suction devices, flush ear cleaning devices, flush anti-colic devices, use as a breast mold injector or other related equipment and functions.

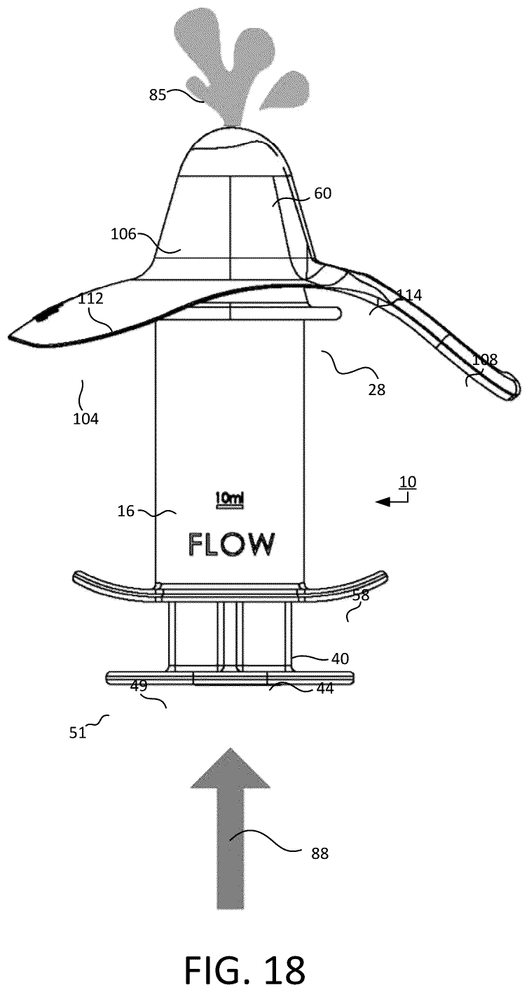

FIGS. 17A and 18 show the flush syringe in the FDS, expelling the fluid 85 from the fluid reservoir 22 and through various channels in the nipple shield 60. When the second external force 88 is applied to the plunger 40, after the fluid reservoir 22 has been filled with the fluid 85, the fluid 85 is forcibly pushed out of the fluid reservoir 22 by the plunger 40. The stopper 42 engages with the fluid 85, translating the compressive forces applied by the second external force 88. In conjunction with the plurality of annular bulges 70 that form a seal with the wall 36, the fluid 85 is forcibly pushed through the outlet 18 and into the channels in the nipple shield 60 at a velocity respective to the second external force 88 applied by a user.

It is to be understood that the fluid 85 stored within the fluid reservoir 22 may be composed of various fluids such as, but is not limited to: water, disinfecting solution, molding liquid, and/or any suitable liquid capable of rinsing, cleaning, flushing and withstanding the applied pressure forces when the plunger 40 is depressed within the barrel 16 to forcibly apply the fluid 85 with a mating device, such as a nipple shield 60, as described in this subject disclosure.

As shown in FIG. 17A, the flush syringe 10 is to be press fit with the nipple shield 60. The concentric barrel projection 34 of the tip 24, engages with an interior surface 110 of a projection 106 of the nipple shield 60 to form a flush cavity 92. The concentric barrel projection 34 is configured such that the outer perimeter of the concentric barrel projection 34 is complementary with a haunch or the dome shaped interior surface 110 of the projection 106 to create a seal, securing the nipple shield 60 to the tip 24 of the flush syringe 10.

During the FDS, as the second external force 88 is applied to the plunger 40 causing the fluid 85 to be pushed through the outlet 18 of the tip 24 into the flush cavity 92. The secure fit between the nipple shield 60 and the tip 24 ensures that the fluid 85 then flows into and through the fluid channel 114 and out of the plurality of openings 116 of the exterior surface 112. As the fluid 85 travels through the fluid channel 114 and out through the plurality of openings 116, the fluid channel 114 is effectively cleaned. The flow of the fluid 85 within the fluid channel 114 may push any debris leftover from use of the nipple shield 60. For example, then the nipple shield 60 is used during breastfeeding, the breastmilk may remain within the fluid channel 114, the flush syringe 10 may then be secured to clean the nipple shield 60 lessening the chances of bacterial build up within the fluid channel 114.

Further, the splash guard 28 may prevent the fluid 85 from escaping the flush cavity 92 and splashing the user if an improper seal is formed between the concentric barrel projection 34 and the nipple shield 60. For example, if the nipple shield 60 and the concentric barrel projection 34 form an improper seal due to improper placement, the splash guard 28 may deflect the fluid that escapes, outwardly away from a user's hand. Moreover, the splash guard 28 may be leveraged by a user when the flush syringe 10 is in use with the nipple shield 60. The splash guard 28 may ensure that the nipple shield 60 is properly sealed to the tip 24 by acting as a guide for the nipple shield 60. For example, a user of the nipple shield 60 may place two fingers on top of the exterior surface 112 of the nipple shield 60 and a thumb underside of the splash guard 28 such that the when the user press fits the nipple shield 60 downwardly onto the tip 24, the concentric barrel projection 34 securely engages with the interior surface 110 of the nipple shield 60 makes. In alternative embodiments, the splash guard 28 may also abut with the nipple shield 60 such that a second seal Is formed between the interior surface 110 of the nipple shield 60 and the splash guard 28.

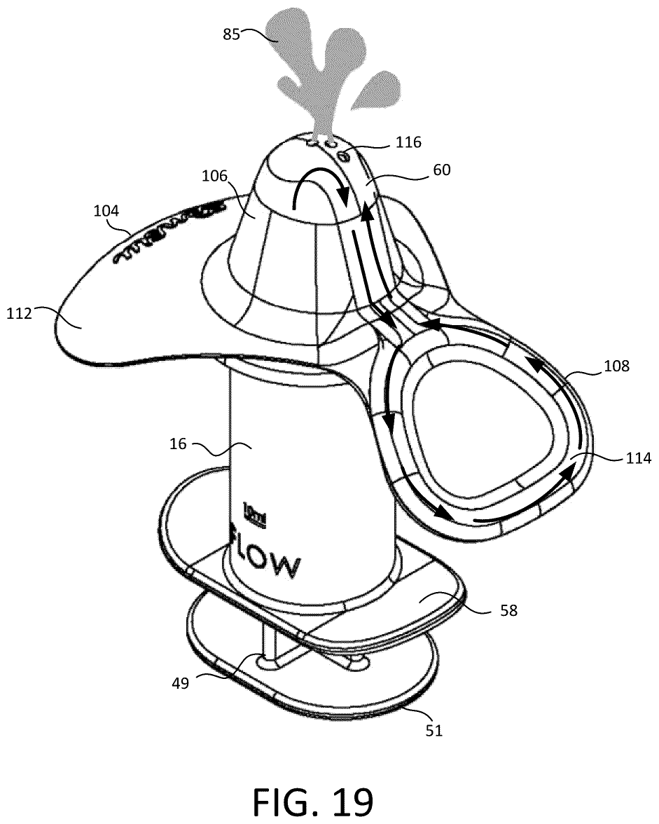

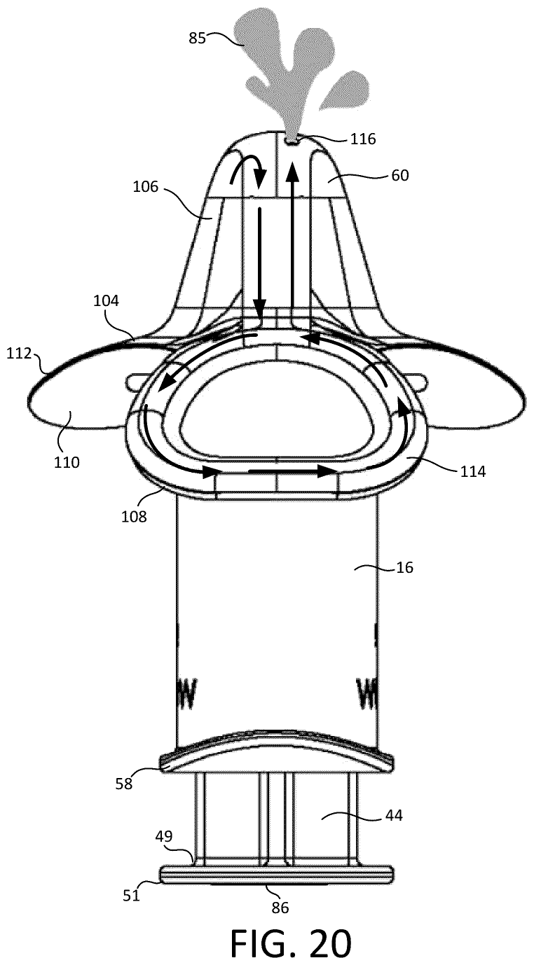

FIGS. 19 and 20 shows the nipple shield 60 has a base portion 104, a projection 106, and a translucent flow indicator 108. The base portion 104 is configured to cover at least a portion of a user's breast. The base portion 104 has an interior surface 110, and an exterior surface 112. The projection 106 has a nipple like shape that concaves upward from the base portion 104. The projection 106 is disposed substantially centrally to the base portion 104, and the translucent flow indicator 108 is disposed away from the projection 106. The translucent visual indicator 108 is defined by a fluid channel 114 connecting the inner surface 110 of the projection 106 with a plurality of openings 116 located on the outer surface 112 at a crown 118 of the projection 106.

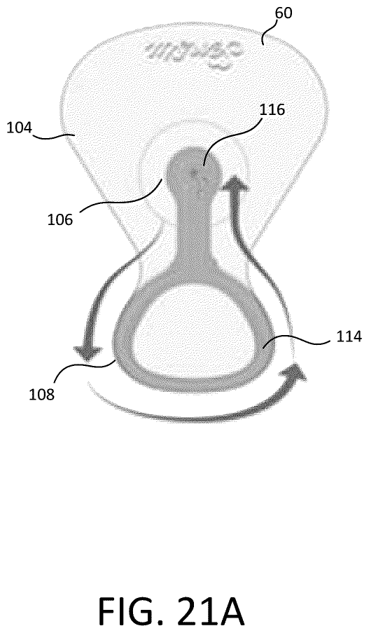

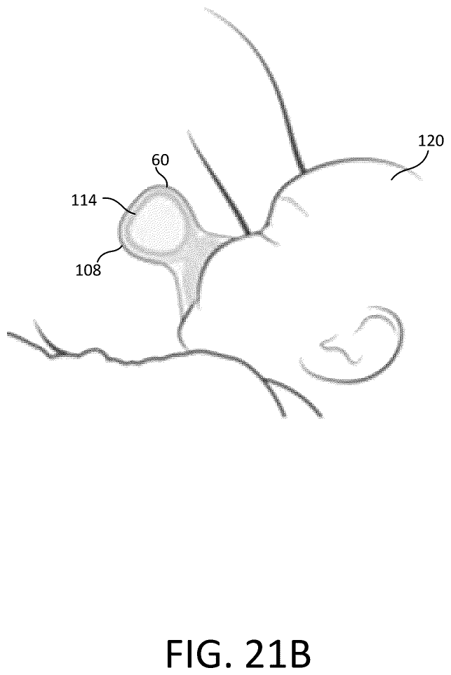

FIGS. 21A and 21B illustrate the translucent flow indicator 108 beginning at and extending the plurality of openings 116 away from the projection 106, such that when the nipple shield 60 is in use, the flow of breast milk from the user can be seen circulating through the translucent flow indicator 108. The nipple shield 60 is made of a translucent pliable material such that the breastmilk can be visibly seen flowing through the fluid channel 114 when the nipple shield 60 is in use. For example, in FIG. 21B the nipple shield 60 is placed over a breast during breastfeeding with an infant, and as the infant 120 suckles from the breast, breastmilk can be seen flowing from the breast through the fluid channel 114 and out into the infants mouth. The translucent flow indicator 108 serves as a visual aid for a breastfeeding mother and provides the benefit of knowing their infant is receiving breastmilk.

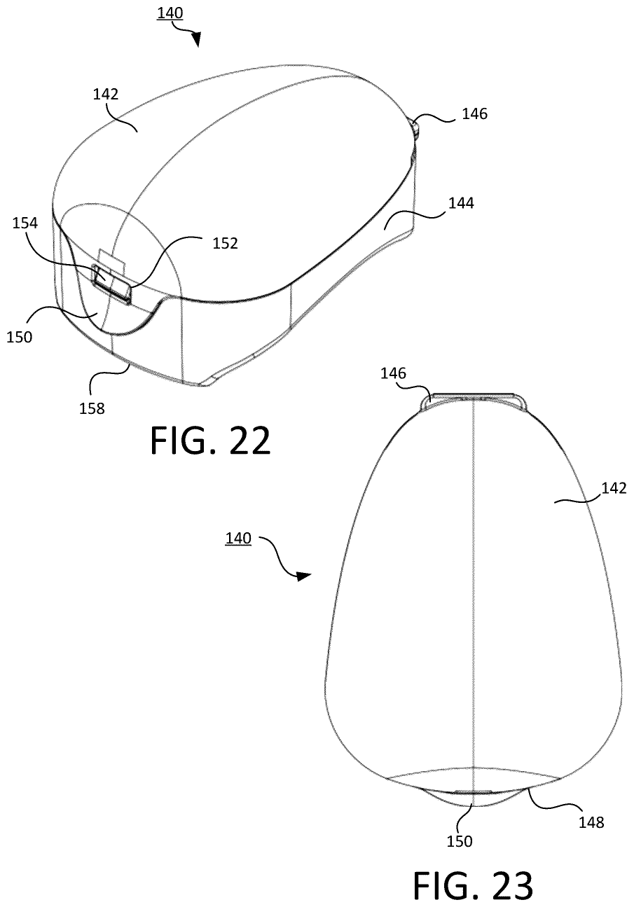

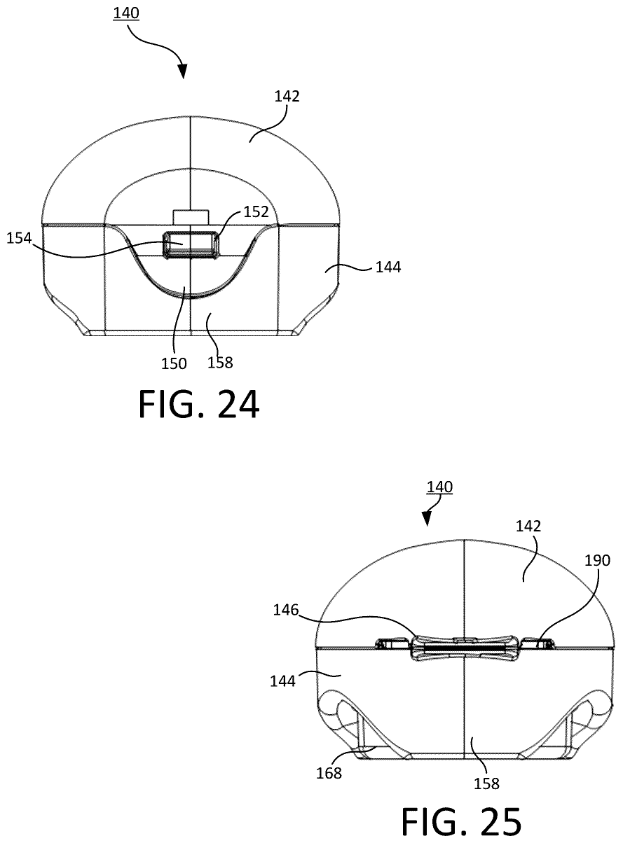

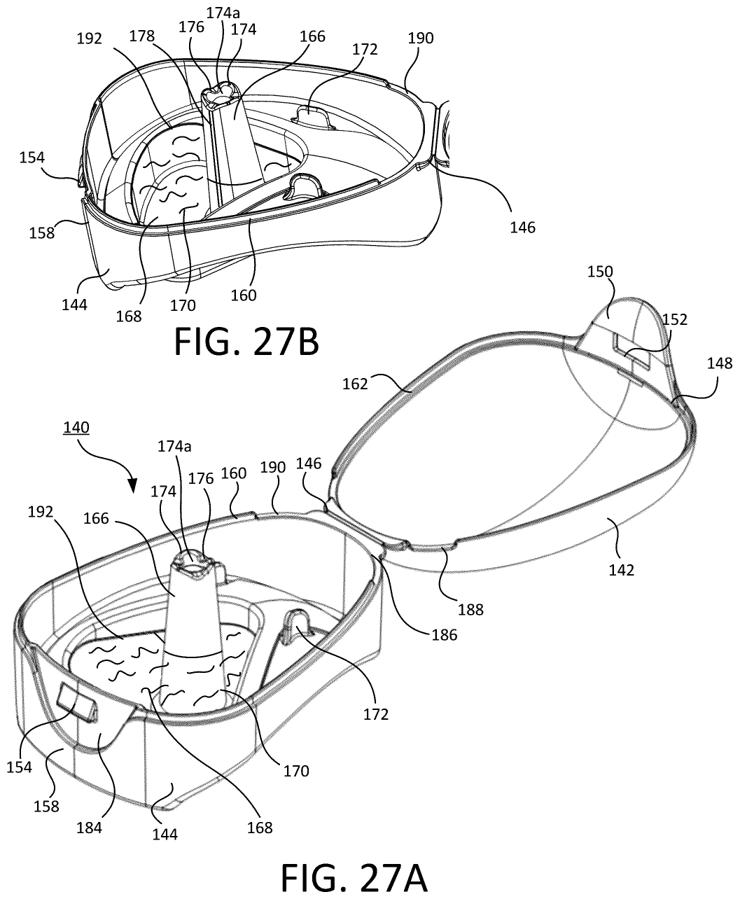

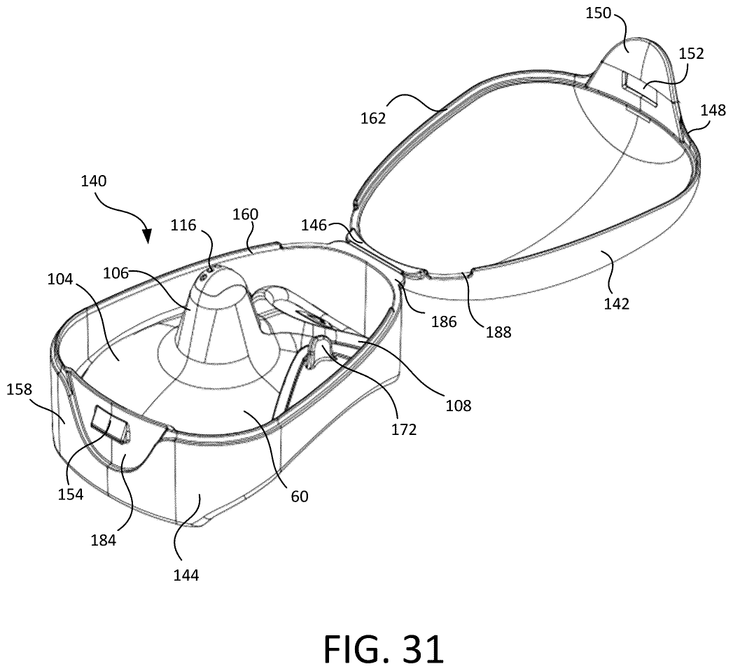

FIGS. 22-25 illustrate the sterilizing case 140. The nipple shield 60 may further be cleaned with a sterilizing case 140. The sterilizing case 140 has a cover 142 hingedly attached by a case hinge 146 to a receptacle 144. As shown in top view, FIG. 23, the cover 142 has an egg shaped exterior capable of enclosing the nipple shield 60. From the case hinge 146, the cover 142 projects uniformly radially outward and downward, then curves in to a case cover end 148. It is to be understood that the cover 142 may have alternative shapes such as, but not limited to a: dome, rectangular, triangular or any other suitable shape that is capable of covering alternate embodiments of the nipple shield 60 of the present disclosure. From the case cover end 148, the cover 142 extends radially down into a finger tab 150. As shown in FIGS. 27A, 27B and 31, the cover 142 has a hallow contoured interior, configured to receive the nipple shield 60.

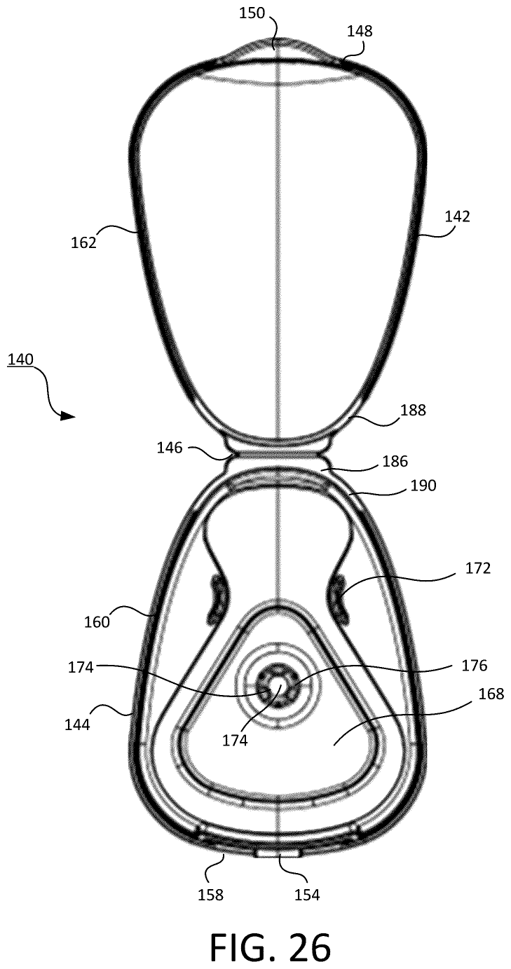

As illustrated in FIGS. 26, 27A, and 27B the finger tab 150 flares outwardly away from the cover 142. The purpose of the finger tab 150 flaring outwardly away from the cover 142 is to provide a user leverage to grip the finger tab 150. Centrally located on the finger tab 150 is a locking aperture 152. The locking aperture 152 is configured to receive a locking protrusion 154. The locking aperture 152 and the locking protrusion 154 together form a case lock 156. When the cover 142 is hingedly closed over the receptacle portion, the finger tab 150 is press fit over the locking protrusion 154, such that the locking protrusion 154 is received within the locking aperture 152, securing the cover 142 to the receptacle 144. The user may then utilize the finger tab 150 to lift the cover over the locking protrusion 154, thus opening the sterilizing case 140. It is to be understood that the case lock 156 may have alternative embodiments such as, but not limited to: magnetic lock, locking indent, clasps or any other suitable locking mechanism capable of securing the cover 142 to the receptacle 144 of the present subject disclosure.

As shown in FIGS. 26 and 27A, and 27B, the receptacle 144 uniformly extends radially outward and downward from the case hinge 146, and then curves in to a receptacle end 158. The upper perimeter of the receptacle 144 further has a recessed edge 160 that encircles the perimeter of the receptacle from a substantially flat segment 186, that is formed by the receptacle 144 portion of the case hinge 146 and the upper section of the receptacle 144, down to the receptacle end 158. At the receptacle end 158 the recessed edge 160 radially extends downward creating a finger tab recess 184 configured to receive the finger tab 150. The locking protrusion 154 is then centrally located to the finger tab recess 184. The recessed edge 160 is configured to receive a mating lip 162 located on the outer perimeter of the cover 142 that encircles the cover 142 from the case hinge 146 and extends into the finger tab 150. Further, there is at least one notch 188 adjacent to the case hinge 146. When the cover 142 is hingedly closed upon the receptacle 144, the recessed edge 160 and the mating lip 162 form a partial seal. Further, the substantially flat segment 186 and the at least one notch 188 form a venting aperture 190. The venting aperture 190 allows generated steam to escape the sterilizing case 140 to de-pressurize the sterilizing case 140 when it is in use. For example, once the partial seal is formed between the cover 142 the receptacle 144, as the temperature of the sterilizing case 140 is raised to generate steam, the pressure will also raise requiring depressurization to which the venting aperture 190 is capable of accomplishing. The venting aperture 190 can also be used as a pour spout in which the fluid 85 within the receptacle 144 can be poured out of without having to open the sterilizing case 140.

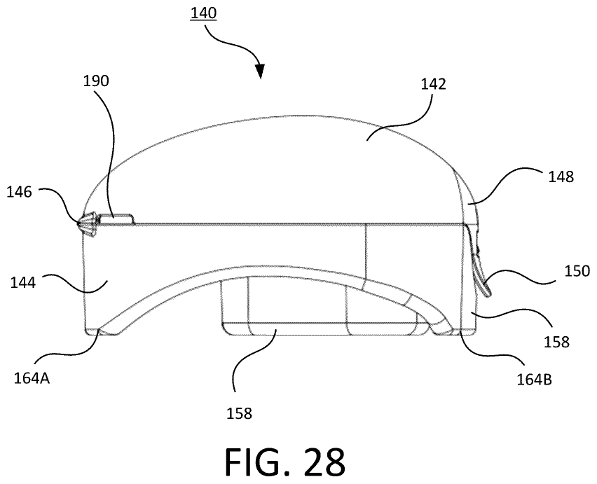

FIG. 28 depicts the receptacle 144 uniformly extending downward from the case hinge 146 to a receptacle leg 164A. From the receptacle leg 164A the receptacle 144 then extends convexly outward into another receptacle leg 164B located at the receptacle end 158. As shown in FIG. 31 the purpose of the convex extension of the receptacle 144 is so that the interior of the receptacle 144 forms an arched support 174 that is configured to receive the nipple shield 60, such that the base portion 104 and the translucent flow indicator 108 of the nipple shield 60 are seated within the receptacle 144.

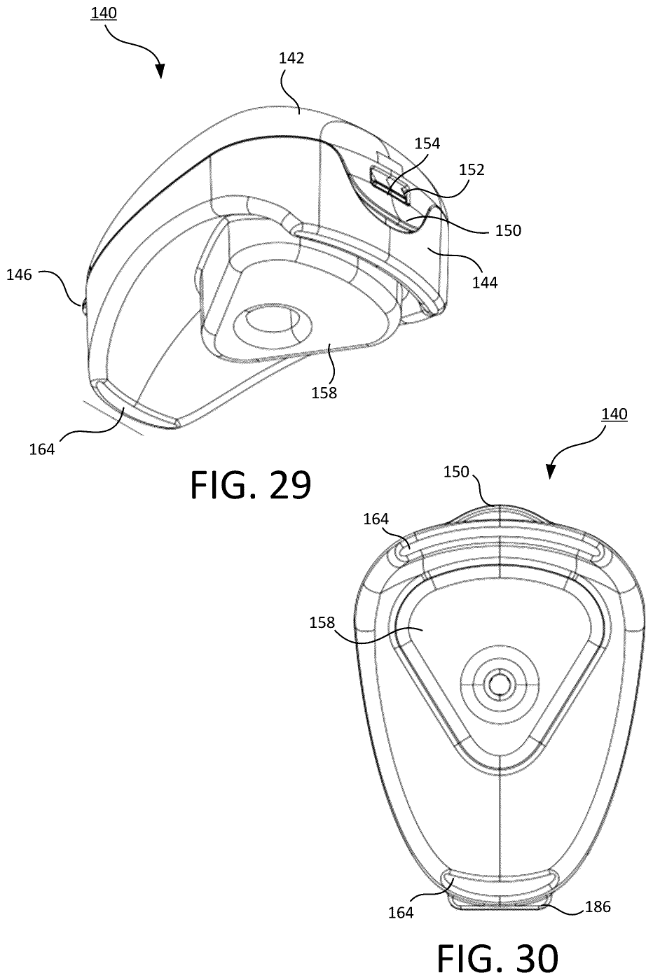

FIGS. 26-31 illustrates the receptacle 144 having a receptacle projection 166 that is substantially centrally located to the receptacle 144. From the bottom of the receptacle 144 the receptacle projection 166 extends conically upward to a receptacle projection tip 174. Referring to FIGS. 27A-27B, the receptacle projection 166 is configured to mate with the projection 106 of the nipple shield 60. The receptacle projection tip 174 has a crenellated configuration. A crenellated configuration, as used herein and throughout this disclosure, is similar to the top notches found at the upper portion of a castle wall. The crenellated configuration or top wall of the castle has upwardly extending walls surrounding a base having alternating sections called “merlons” (i.e., the solid parts) and “crenels” (i.e., the openings). Similarly, the projection tip 174 may have a recessed base wall 174a within the projection tip 174. The slightly recessed base wall 174a of the projection tip is surrounded by a crenellated-like wall formed of solid portions and openings like the top of a castle. The receptacle projection 166 with projection tip 174 may also resemble a chimney stack with an uneven top portion. The crenellated-like wall may resemble a scalloped wall or similar defining a plurality of openings or notches 176 through which the flow of steam is permitted to flow directly across the opening notches 176 into a centermost portion over the base wall 174a of the projection tip 174 and directly into openings in the nipple shield 60, as will be discussed in greater detail later in this subject disclosure. It is to be understood, that the projection tip 174 may have at least one solid part and one opening. The benefits of the plurality of notches 176 is to overcome any seal that may form between the receptacle projection 166 and the projection 106 of the nipple shield 60. It is to be understood that the receptacle projection tip 174 may have alternative configurations such as, but not limited to: rounded tip, singular notch, or any other suitable configuration that is capable of mating with the projection 106 of the nipple shield 60. Further, it is to be understood that the receptacle projection 166 may have alternative embodiments such as but not limited to: cylindrically shaped, dome shaped, rectangular shaped or any other suitable configuration capable of receiving the projection 106 of the nipple shield 60.

Referring back to FIGS. 27B, the receptacle projection 166 may also have at least one channel 178 that runs from the base of the receptacle projection 166 up to the receptacle projection tip 174. The at least one channel 178 may be equally disbursed throughout the perimeter of the receptacle projection 166. Further, the receptacle projection 166 may be removably secured to the receptacle 144 through one of any suitable fastening means such as but limited to: press fitting, mating magnets, clasps or any other suitable fastening method capable of removably securing the receptacle projection 166 to the receptacle 144. It is to be understood that that the receptacle projection 166 may also be molded to the receptacle 144. The receptacle projection 166 may also have an at least one internal channel 180 that is centrally bored through the receptacle projection 166. The at least one internal channel 180 may have at least one channel inlet 182 located at the base of the receptacle projection 166 that is in communication with the at least one internal channel 180. The at least one internal channel 180 may permit generated steam to flow from the at least one channel inlet 182, through the at least one internal channel 180 and into the nipple shield 60 as will be discussed in further detail later in this subject disclosure.

FIGS. 27A-27B, depicts the receptacle projection 166 is encircled by a trough or receptacle reservoir 168 that begins from the upper midsection of the receptacle 144 and radially expands outward and downward toward the receptacle end 158 and incurves to enclose the receptacle reservoir 168. The recessed reservoir 168 is configured to receive a cleaning liquid 170 such as but not limited to: water, soap, or any other suitable liquid that is capable of cleaning, rinsing, and/or sterilizing the nipple shield 60 a described in this subject disclosure. At a predetermined segment of the receptacle reservoir 168, a receptacle fill line indicator 192 encircles the perimeter of the receptacle reservoir 168. The receptacle fill line indicator 192 serves as a visual indicator for the user to show how much of the cleaning liquid 170 is required for use of the sterilizing case 140.

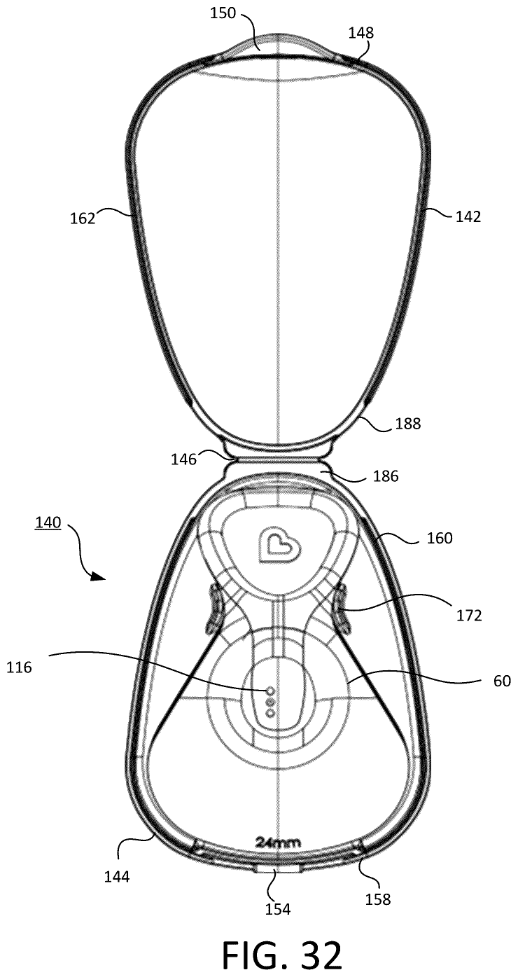

FIGS. 27A, 27B and 31-32 depict the receptacle 144 having two crescent shaped projections 172 located at an intermediate surface above the receptacle reservoir 168. The nipple shield 60 is seated and aligned within the receptacle 144 where the projection 106 is atop the receptacle projection 166 and the two crescent shaped projections 172 receive the upper portion of the translucent flow indicator 108. The benefits of the two crescent shaped projections 172 are to stabilize the nipple shield 60 when it is seated within the receptacle 144. For example, when the nipple shield 60 is enclosed by the cover 142, the crescent shaped projections 172 align and stabilize the nipple shield 60 and mitigate lateral movement of the nipple shield 60 when the sterilizing case 140 is moved so as to prevent the soft nipple shield 60 from being damaged. The two crescent shaped projections 350 further may serve as a guide for the user to properly insert the nipple shield 60 within the receptacle 144. For example, the user may use the two crescent shaped projections 172 to ensure that the nipple shield 60 is properly inserted into the receptacle 144 because the two crescent shaped projection 172 coincide with a tapered portion of the nipple shield 60, such that the translucent flow indicator 108 is oriented above the base portion 104 of the nipple shield 60, as shown in FIG. 31-32. It is to be understood that the two crescent shaped projections 172 may have alternative embodiments such as but not limited to: cylindrical shape, rectangular shape, triangular shape, circular shape or any other suitable shape capable of stabilizing the nipple shield 60 or equivalent infant device as described in the subject disclosure.

The operation of the sterilizing case 140 is provided as follows: a user opens the sterilizing case 140 by lifting the cover 142 to an open configuration as shown in FIGS. 26, 27A and 27B. The user then pours into the receptacle reservoir 168 the cleaning liquid 170 up to the receptacle fill line indicator 192. The user then places the nipple shield 60 within the receptacle as shown in FIGS. 31-32 and then closes the sterilizing case 140 by hingedly covering the receptacle 144 with the cover 142 ensuring that the case lock 156 is engaged. The user may then place the sterilizing case 140 in a heating device, such as but not limited to a microwave, to bring the cleaning liquid 170 to boiling temperature such that the cleaning liquid 170 converts to steam.

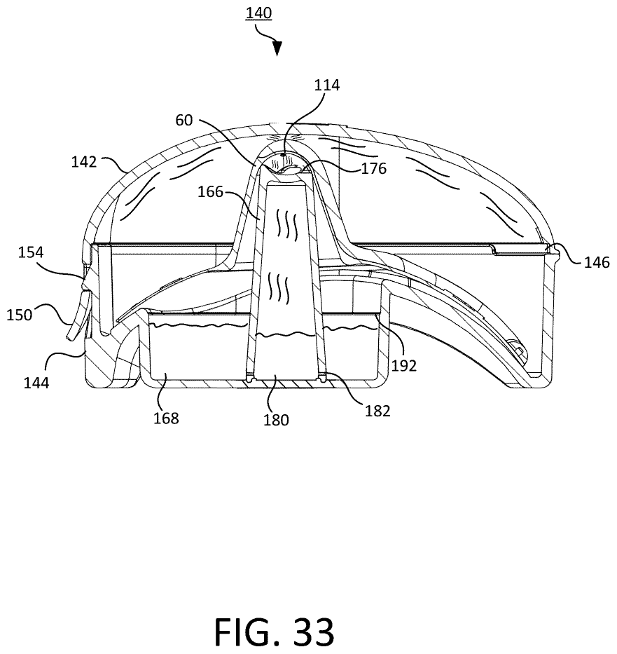

FIG. 33 illustrates the generated steam tracking upward from the receptacle reservoir 168, up the receptacle projection 166 and then enters the nipple shield 60 through the plurality of notches 176. The steam then covers all surfaces of the nipple shield 60, as well as enters the fluid channel 114 of the nipple shield 60, then through the translucent flow indicator 108 and exits from the plurality of openings 116, whereby the nipple shield 60 is effectively rinsed and sterilized. For example, the steam may push any residue left behind within the fluid channel, such as breast milk, from the nipple shield 60. The combination of the heat and possible sterilizing capability of the cleaning liquid 170, sterilize the nipple shield 60 for continued safe usage. In alternative embodiments where the sterilizing case 140 has the at least one channel 178 on the receptacle projection 166, or the at least one internal channel 180 within the receptacle projection 166, the steam may be guided by the at least one channel 178 or enter the at least one internal channel 180 from the at least one channel inlet 182 upward from the receptacle reservoir 168 and into the fluid channel 114. It is to be understood that the sterilizing case 140 may have any combination of the plurality of notches, the at least one channel 178 and/or the at least one internal channel 180, suitably capable of directing the flow of steam from the receptacle reservoir 168 upward into the nipple shield 60 or equivalent mating device.

The sterilizing case 140 may also be placed into a container having hot water or equivalent liquid such that the cleaning liquid 170 may convert into steam as described by the present subject disclosure. For example, the sterilizing case 140 may be placed into a pot of boiling water capable of raising the temperature of the cleaning liquid 170 to generate steam. Furthermore, it is to be understood that in alternative embodiments the material characteristics of the sterilizing case 140 may include, but are not limited to: metal, plastic, polymers, or any other material suitably capable of withstanding the temperature needed raise the temperature of the cleaning liquid 170 to generate the steam as described in this subject disclosure.

The sterilizing case 140 may be used to sterilize other infant related products such as but limited to: pacifiers, bottle nipples, teething toys or any other infant products that may have crevices that require rinsing, cleaning and/or sterilizing that can fit within the sterilizing case 140 as described by the present subject disclosure. Furthermore, the sterilizing case 140 may be used to store and safe keep said related items, such as a nipple shield, for traveling purposes or to keep exposure to the atmosphere limited.

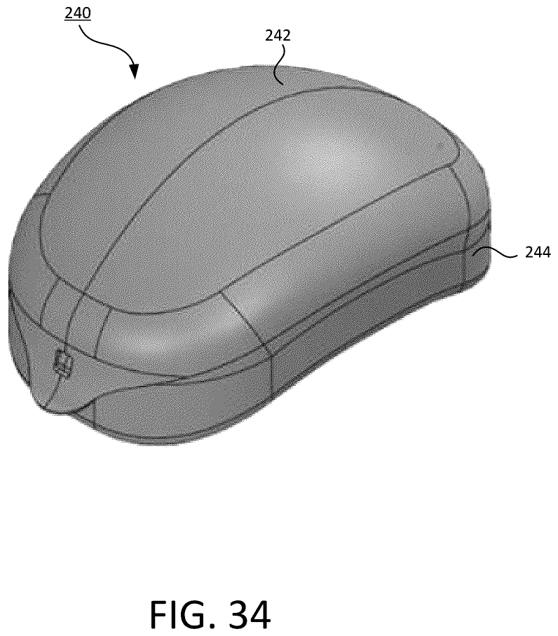

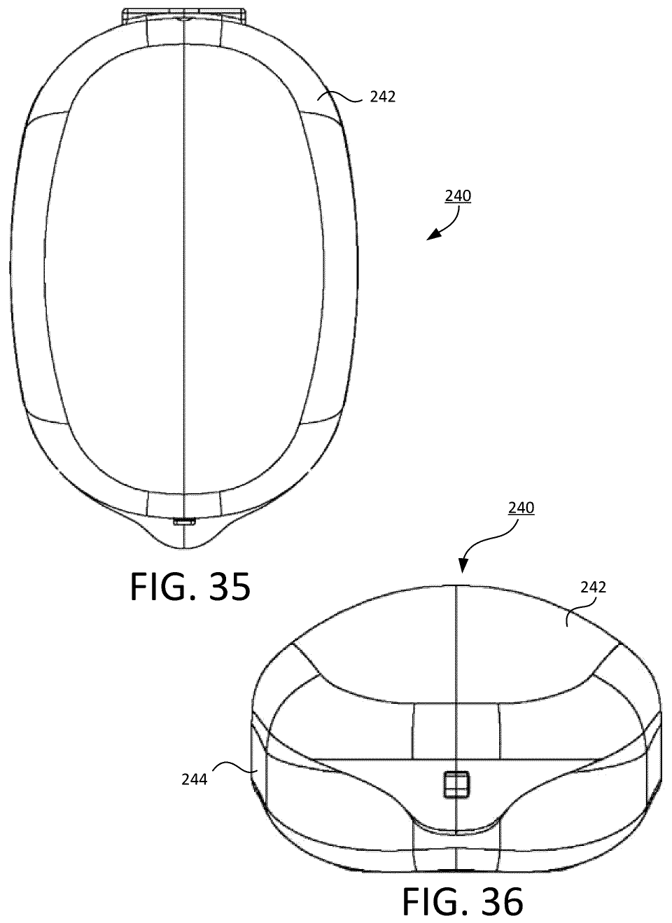

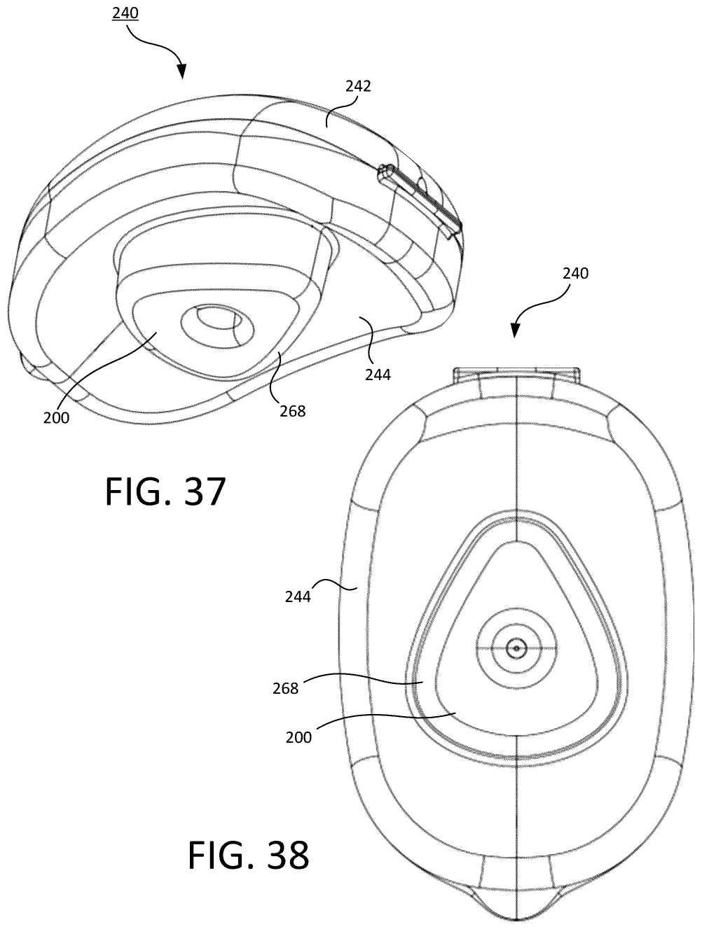

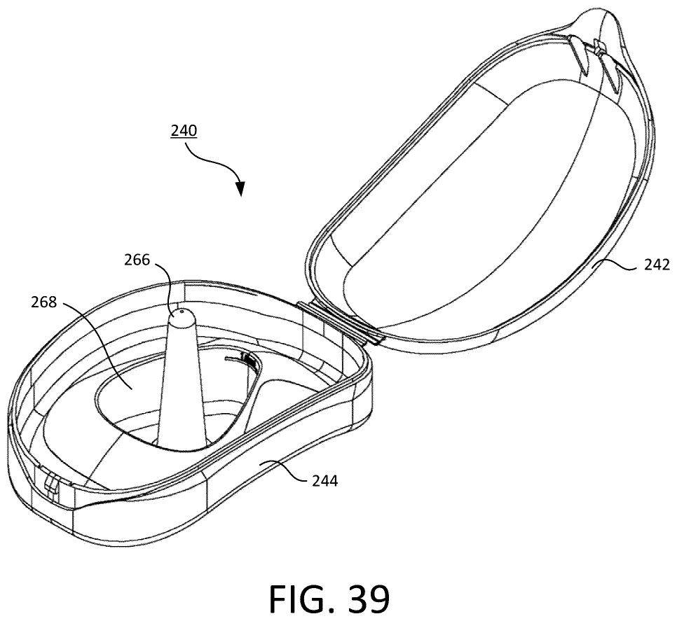

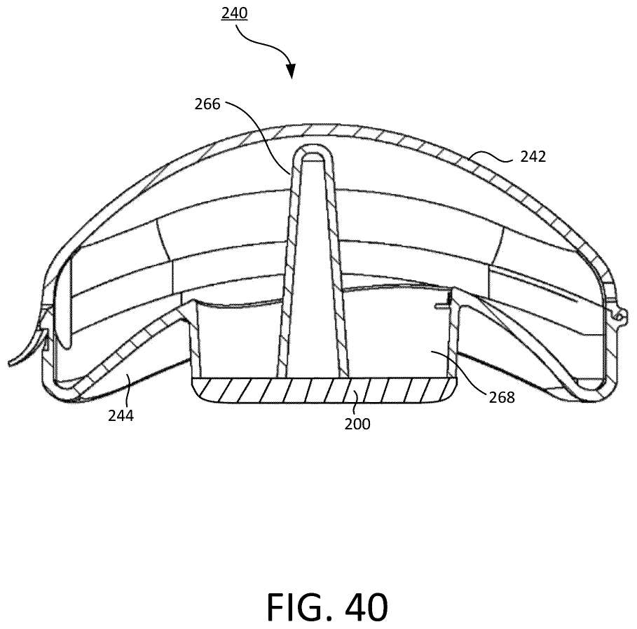

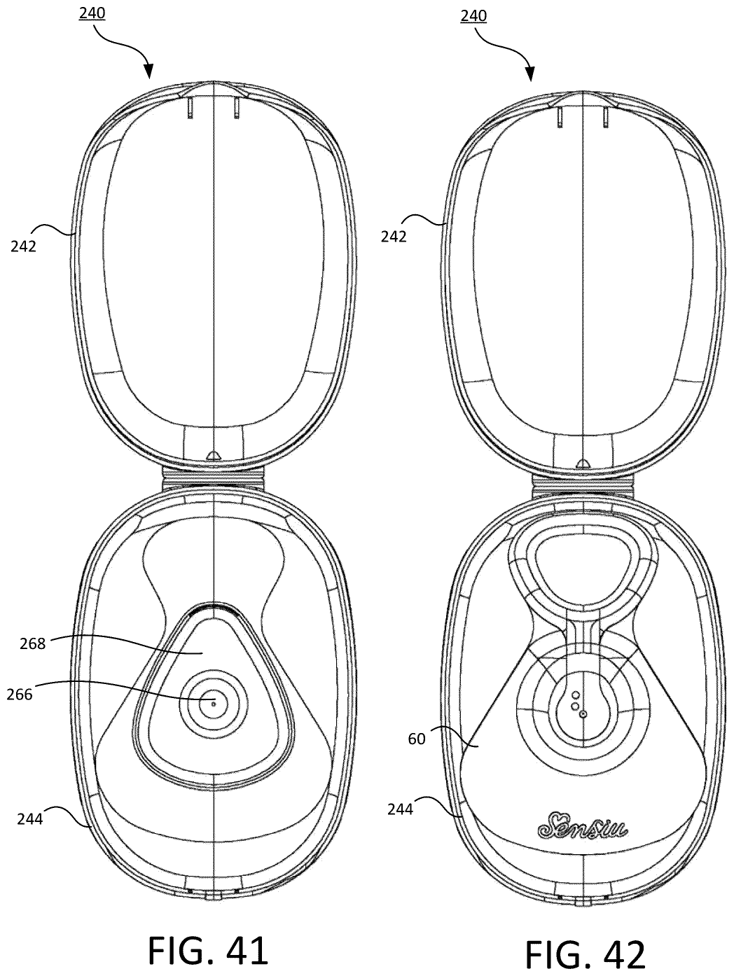

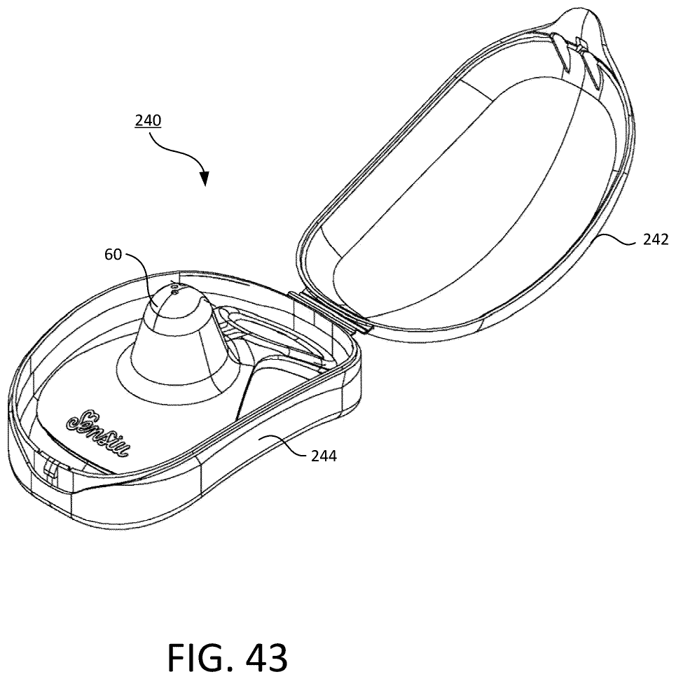

FIGS. 34-43 illustrates another embodiment for a sterilizing case 240 which may have alternate embodiments that are capable of performing the features as described in the present subject disclosure. As shown, the sterilizing case 240 may take on a variety of different shapes and constructions. The sterilizing case 240 is more soap-bar shaped having an upper curved top cover 242, a lower curved receptacle 244 with a receptacle reservoir 268 disposed therein. Likewise, a receptacle projection 266 is provided to align and secure the nipple shield 60 during storage and cleaning. As shown in FIG. 40, in other alternate embodiments, the receptacle reservoir may be configured to include a heating plate 200 that is capable of generating steam out of the cleaning liquid 170. The benefits of including a heating plate would permit the user portability when traveling to safe keep the nipple shield 60 or other products within the sterilizing case 140, while also having the ability to sterilize the products.

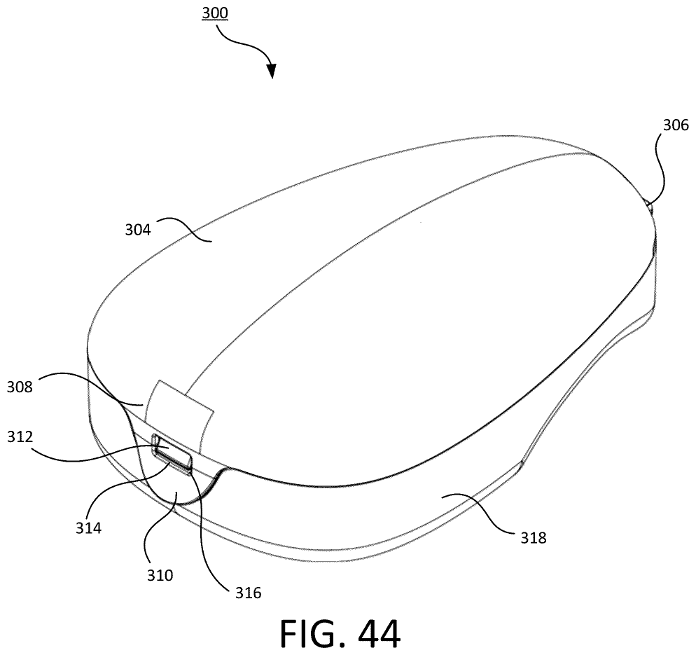

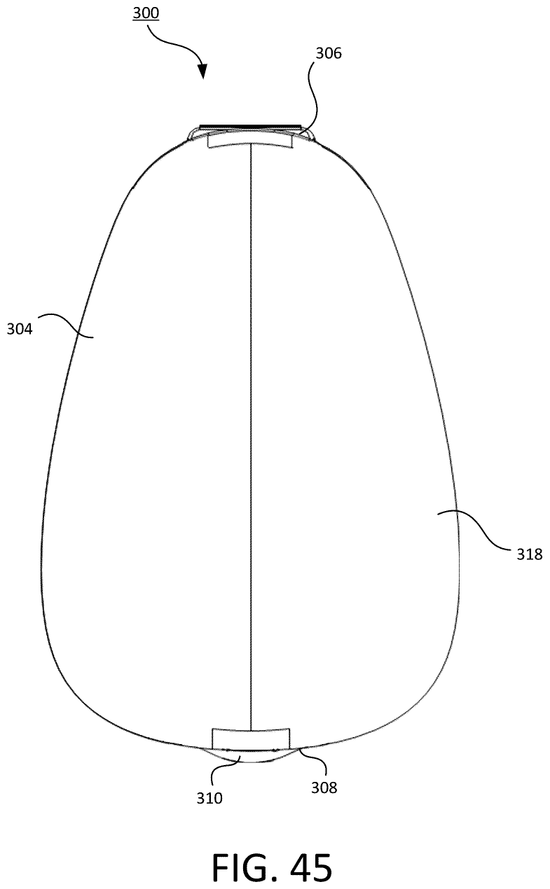

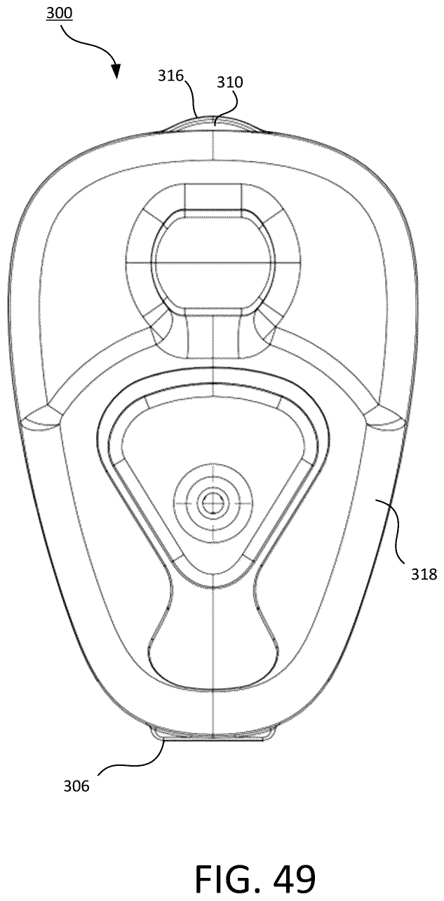

FIGS. 44-49 show an embodiment for a sterilizing storage case 300. The sterilizing storage case 300 is constructed to house both the flush syringe 10 and the nipple shield 60 together and/or sterilized by the sterilizing storage case 300. The sterilizing storage case 300 has a sterilizing storage case cover 304 that projects from a sterilizing storage case hinge 306 uniformly, radially outward and downward. The sterilizing storage case cover 304 then curves in to a sterilizing storage case end 308. The sterilizing storage case cover 304 has pear-shape with a solid exterior and hollow interior and is configured to hingedly enclose the nipple shield 60 and flush syringe 10 within a sterilizing storage case receptacle 318. It is to be understood that the sterilizing storage case cover 304 may have alternative shapes such as, but not limited to a: dome, rectangular, triangular or any other suitable shape that can cover alternate embodiments of the nipple shield 60 and flush syringe 10 of the present disclosure. From the sterilizing storage case end 308, the sterilizing storage case cover 304 extends radially down into a tab 310.

The tab 310 flares outwardly away from the sterilizing storage case cover 304. The purpose of the tab 310 flaring outwardly away from the sterilizing storage case cover 304 is to provide a user leverage in order to grip the tab 310. Centrally located on the tab 310 is a sterilizing storage case locking aperture 312. The sterilizing storage case locking aperture 312 is configured to receive a sterilizing storage case locking protrusion 314 located on a sterilizing storage case receptacle end 320. The sterilizing storage case locking aperture 312 and the sterilizing storage case locking protrusion 314 together form a sterilizing storage case lock 316. When the sterilizing storage case cover 304 is hingedly closed over the sterilizing storage case receptacle 318, the tab 310 is press fit over the sterilizing storage case locking protrusion 314, such that the sterilizing storage case locking protrusion 314 is received within the sterilizing storage case locking aperture 312, securing the sterilizing storage case cover 304 to the sterilizing storage case receptacle 318. The user may then utilize the tab 310 to lift the sterilizing storage case cover 304 over the sterilizing storage case locking protrusion 314, thus opening the sterilizing storage case 300. It is to be understood that the sterilizing storage case lock 316 may have alternative embodiments such as, but not limited to: magnetic lock, locking indent, clasps or any other suitable locking mechanism capable of securing the sterilizing storage case cover 304 to the sterilizing storage case receptacle 318 of the present subject disclosure.

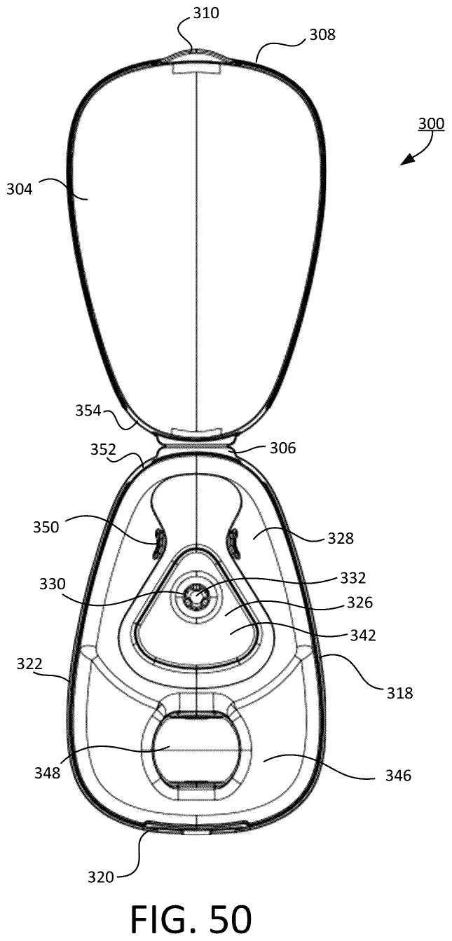

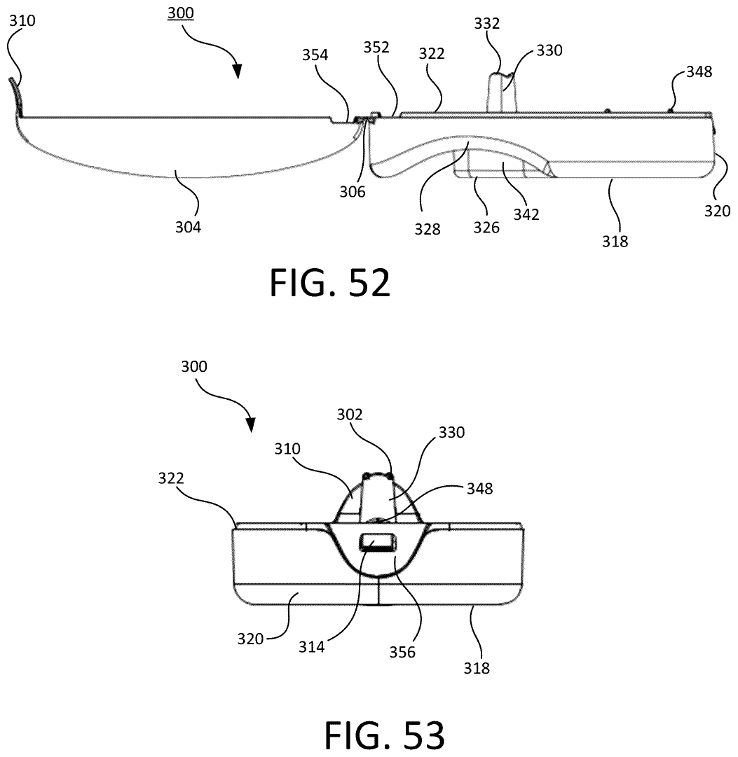

FIGS. 50-53 depict the sterilizing storage case receptacle 318 uniformly extending radially outward and downward from the sterilizing storage case hinge 306, and then incurves to the sterilizing storage case receptacle end 320. The upper perimeter of the sterilizing storage case receptacle 318 has a recessed edge 322 that encircles the perimeter of the sterilizing storage case receptacle 318 from the sterilizing storage case hinge 306 down to the sterilizing storage case receptacle end 320.

At least one receptacle notch 352 is adjacent to the sterilizing storage case hinge 306. At the sterilizing storage case receptacle end 320 the recessed edge 322 extends radially downward creating a sterilizing storage case tab recess 356 configured to receive the tab 310. The sterilizing storage case locking protrusion 314 is then centrally located to the sterilizing storage case tab recess 356. The recessed edge 322 is configured to receive a mating lip 324 located on the outer perimeter of the sterilizing storage case cover 304 that encircles the sterilizing storage case cover 304 from the sterilizing storage case hinge 306 and extends into the tab 310.

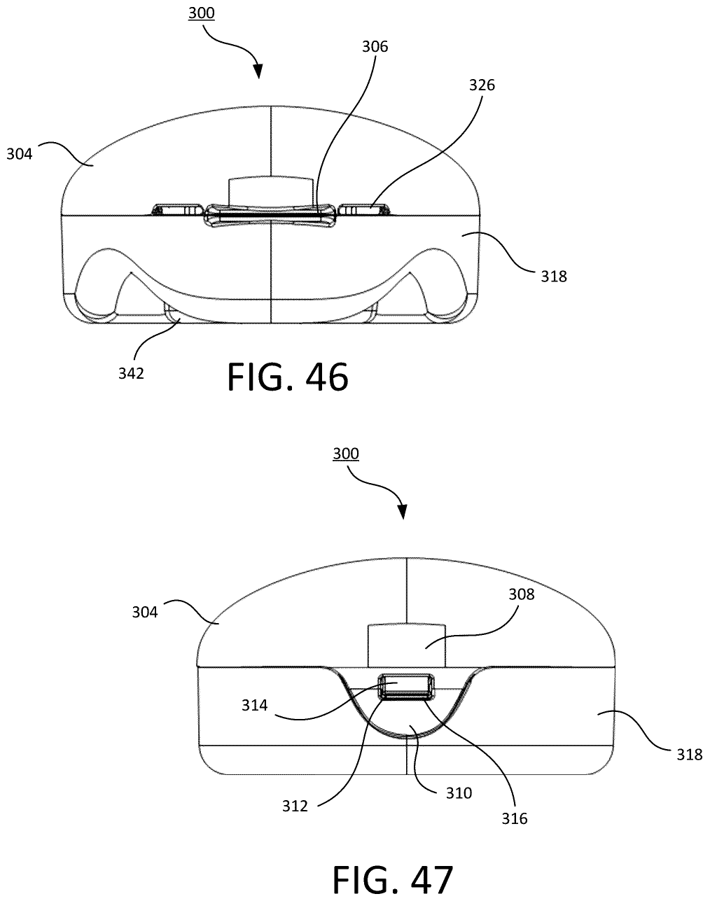

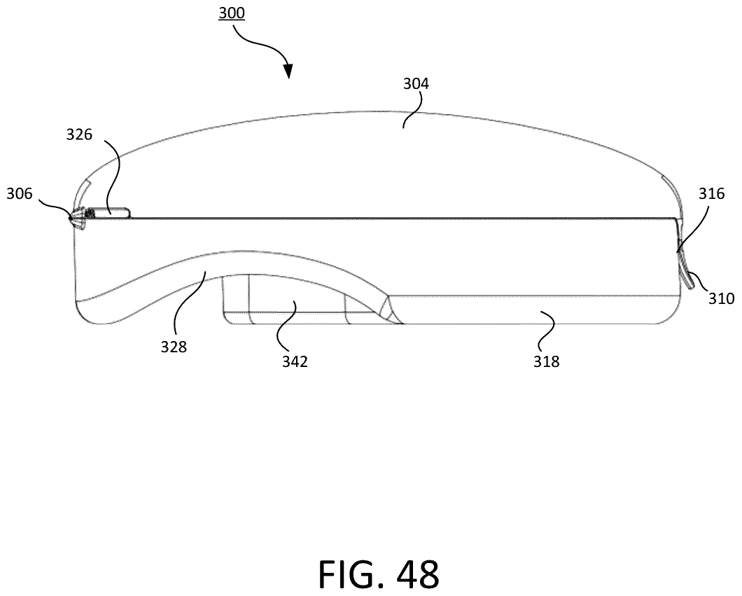

FIGS. 46 and 48 depict an at least case cover notch 354 adjacent to the sterilizing storage case hinge 306. When the sterilizing storage case 300 is hingedly closed upon the sterilizing storage case receptacle 318, the recessed edge 322 and the mating lip 324 form a seal. Further, the at least one receptacle notch 352 and the at least one case cover notch 354 form a venting aperture 326. The venting aperture 326 allows generated steam to escape the sterilizing storage case 300 to de-pressurize the sterilizing storage case 300 when it is in use. For example, when the sterilizing storage case cover 304 is hingedly closed upon the sterilizing storage case receptacle 318, the mating lip 324 sits within the recessed edge 322 and in conjunction with the sterilizing storage case lock 316, the contents of the sterilizing storage case 300 are sealingly enclosed within, as the temperature of the sterilizing storage case 300 is raised to generate steam the pressure will equally raise requiring depressurization to which the venting aperture 326 is capable of allowing the release of the pressure.

The sterilizing storage case receptacle 318 projects downward from the recessed edge 322 to a sterilizing storage case receptacle base 326. The upper portion of the sterilizing storage case receptacle base 326 has a convex portion to form an arched support 328 that is configured to receive the nipple shield 60, such that the base portion 104 and the translucent flow indicator 108 of the nipple shield 60 are seated within the sterilizing storage case receptacle 318.

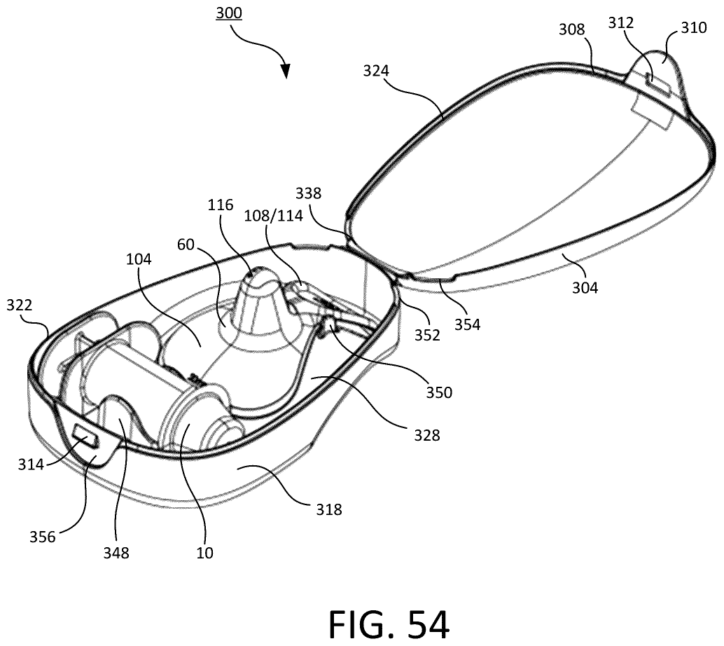

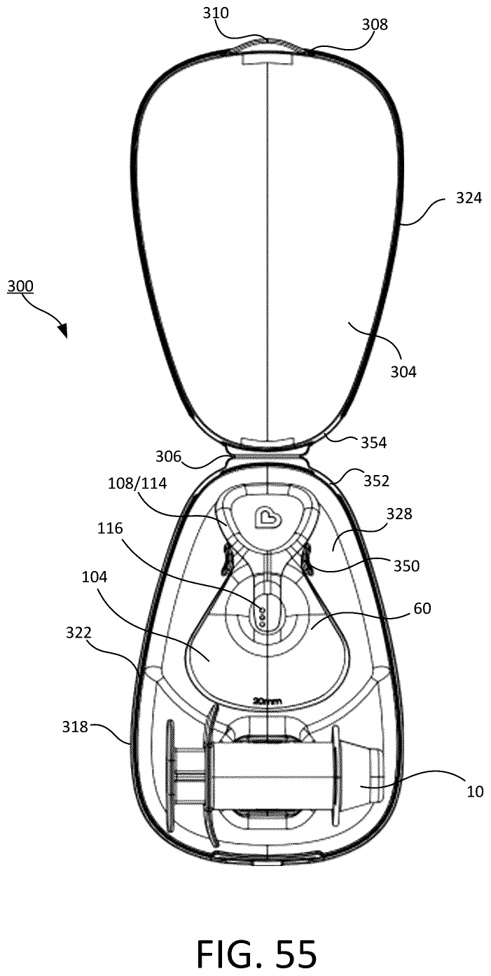

The sterilizing storage case receptacle 318 has two crescent shaped projections 350 located at the upper midsection of the sterilizing storage case receptacle 318. The two crescent shaped projections 350 are configured to secure and/or stabilize the nipple shield 60 as will be discussed in further detail later in this subject disclosure. The two crescent shaped projections 350 further may serve as a guide for the user to properly insert the nipple shield 60 within the sterilizing storage case receptacle 318. For example, the user may use the two crescent shaped projections 350 to ensure that the nipple shield 60 is properly inserted into the sterilizing storage case receptacle 318 because the two crescent shaped projection 318 coincide with a tapered portion of the nipple shield 60, such that the translucent flow indicator 108 is oriented above the base portion 104 of the nipple shield 60, as shown in FIGS. 54-55. It is to be understood that the two crescent shaped projections 350 may have alternative embodiments such as but not limited to: cylindrical shape, rectangular shape, triangular shape, circular shape or any other suitable shape capable of stabilizing the nipple shield 60 or equivalent infant device as described in the subject disclosure.

Further, the sterilizing storage case receptacle 318 has a receptacle projection 330 that is centrally located to the arched support 328. The receptacle projection 330 conically extends upward from the sterilizing storage case receptacle base 326 and then narrows inward to a receptacle projection tip 332. The receptacle projection 330 is configured to mate with the projection 106 of the nipple shield 60.

The receptacle projection tip 332 has a plurality of notches 302 to direct the flow of steam into fluid channel 114 of the nipple shield 60 as will be discussed in greater detail later in this subject disclosure. It is to be understood that the receptacle projection tip 332 may have alternative configurations such as, but not limited to: triangular tip, oblong tip, rectangular tip, or any other suitable configuration that is capable of mating with the projection 106 of the nipple shield 60. Further, it is to be understood that the receptacle projection 330 may have alternative embodiments such as but not limited to: cylindrically shaped, dome shaped, rectangular shaped or any other suitable configuration capable of receiving the projection 106 of the nipple shield 60.

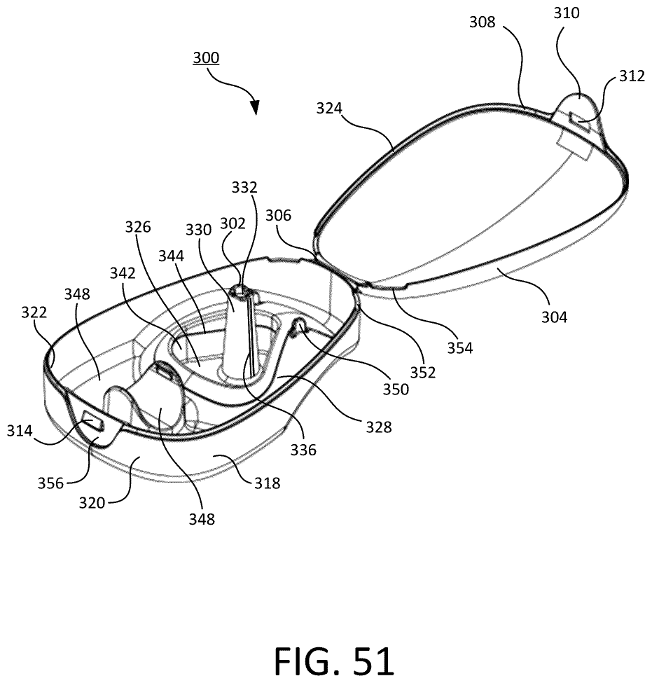

Referring to FIG. 51, the receptacle projection 330 may also have at least one of a channel 336 that runs from the sterilizing storage case receptacle base 326 of the receptacle projection 330 up to the receptacle projection tip 332. The one channel 336 may be equally disbursed throughout the perimeter of the receptacle projection 330. The one channel 336 may be bored on the exterior of the receptacle projection 330. Further, the receptacle projection 330 may be removably secured to the sterilizing storage case receptacle 318 through one of any suitable fastening means such as but limited to: press fitting, mating magnets, clasps or any other suitable fastening method capable of removably securing the receptacle projection 330 to the sterilizing storage case receptacle 318. It is to be understood that that the receptacle projection 330 may also be molded to the sterilizing storage case receptacle 318.

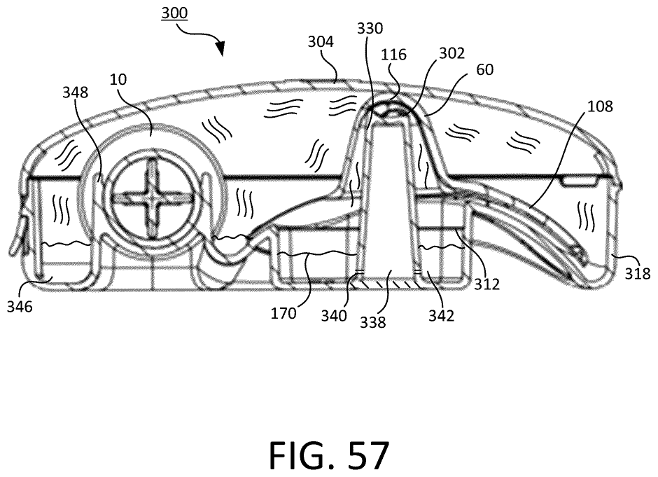

FIG. 57 illustrates the receptacle projection 330 may also have an at least one internal channel 338 that is centrally bored through the receptacle projection 330. The internal channel 338 may also have at least one channel inlet 340 located at the base of the receptacle projection 330 that is in communication with the at least one internal channel 338. The one internal channel 338 may permit generated steam to flow from the one channel inlet 340, through the one internal channel 338 and into the nipple shield 60 as will be discussed in further detail later in this subject disclosure.

The receptacle projection 330 is enclosed by a receptacle reservoir 342 that begins from the upper midsection of the sterilizing storage case receptacle 318 and radially extends outward and downward toward the sterilizing storage case receptacle end 320 and curves in to form the receptacle reservoir 342. The receptacle reservoir 342 is configured to receive a cleaning liquid 170 such as but not limited to: water, soap, or any other suitable liquid that is capable of cleaning, rinsing, and/or sterilizing the nipple shield 60 a described in this subject disclosure. At a predetermined height of the receptacle reservoir 342, a receptacle fill line indicator 312 encircles the perimeter of the receptacle reservoir 342. The receptacle fill line indicator 312 in the receptacle reservoir 342 serves as a visual indicator for the user to show how much of the cleaning liquid 170 is required for cleaning use of the sterilizing storage case 300.

From the upper portion of the sterilizing storage case receptacle base 326 the sterilizing storage case receptacle base 326 then lowers into a receptacle basin 346 located approximately central to the sterilizing storage case receptacle base 326. That is, centrally located to the receptacle basin 346 is a parabolic projection 348 that is configured to receive the flush syringe 10. The parabolic projection 348 extends upward from the sterilizing storage case receptacle base 326 into two equally spaced flanges.

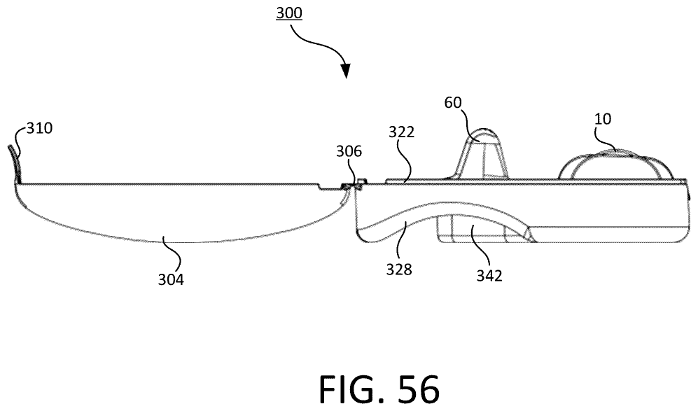

FIGS. 54-56 depict when the flush syringe 10 is inserted into the parabolic projection 348, the parabolic projection 348 secures and/or stabilizes the flush syringe 10 when the sterilizing storage case 300 is in a storage position. For example, when the sterilizing storage case 300 is closed with the flush syringe 10 secured by the parabolic projection 348, the parabolic projection 348 limits the lateral movement of the flush syringe 10 when the sterilizing storage case 300 is moved about. The nipple shield 60 is seated within the sterilizing storage case receptacle 318 where the projection 106 is located atop the receptacle projection 330 and the crescent shaped projections 350 receive the upper portion of the translucent flow indicator 108 of the nipple shield 60. The benefits of the crescent shaped projections 350 are to stabilize the nipple shield 60 when it is seated within the sterilizing storage case receptacle 318. For example, when the nipple shield 60 is enclosed by the sterilizing storage case cover 304, the crescent shaped projections 350 stabilize the nipple shield 60 and mitigate lateral movement of the nipple shield 60 when the sterilizing storage case 300 is moved.

The sterilizing storage case 300 permits the user to both store and sterilize the nipple shield 60 and the flush syringe 10. In the storage configuration, the user simply lifts the sterilizing storage case cover 304 to an open configuration and inserts and aligns the nipple shield 60 over the receptacle projection 330 and between the two crescent shaped projections 350. The user then places the flush syringe 10 within the parabolic projection 348 and then closes the sterilizing storage case cover 304 over the sterilizing storage case receptacle 318, ensuring that the sterilizing storage case lock 316 is engaged. The user then may proceed to travel with the sterilizing storage case 300, with the nipple shield 60 and flush syringe 10 secured therein.

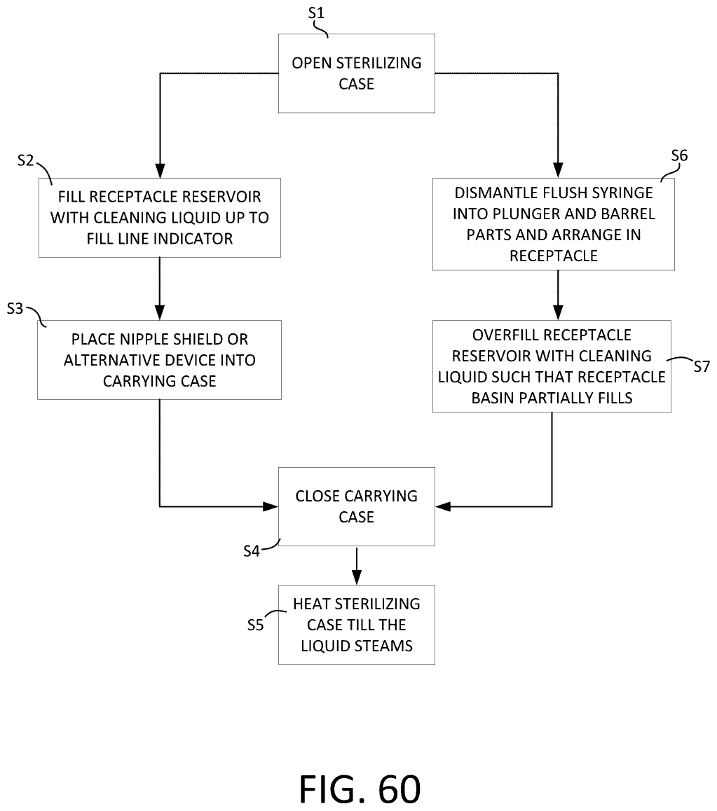

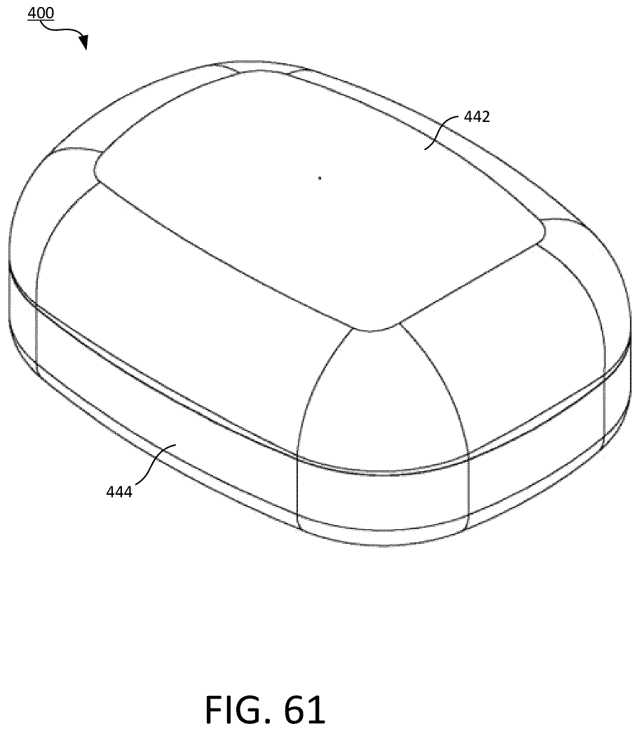

FIG. 60 illustrates an exemplary method for cleaning the nipple shield 60 and flush syringe 10 within the sterilizing storage case 240, 300, 400. In step S1, open the sterilizing storage case. Proceed to step S2.

In step S2, fill the receptacle reservoir with the cleaning liquid up to the receptacle fill line indicator. Proceed to step S3.

In step S3, place the nipple shield 60 or alternative device into the sterilizing storage case. An alternative device could be for example a pacifier, and/or any other device capable of being cleaned within the sterilizing storage case. Proceed to step S4.

In step S4, close the sterilizing storage case with the nipple shield and alternative devices arranged within. Proceed to step S5.

In step S5, place the sterilizing storage case within a heating device and proceed to heat the sterilizing storage case until it comes to a boil turning the cleaning liquid into a steam that can then it transfer into the nipple shield 60 sterilizing the fluid channel.

Alternatively, if the sterilizing storage case includes the nipple shield 60 and the flush syringe, after step S1, proceed to step S6.

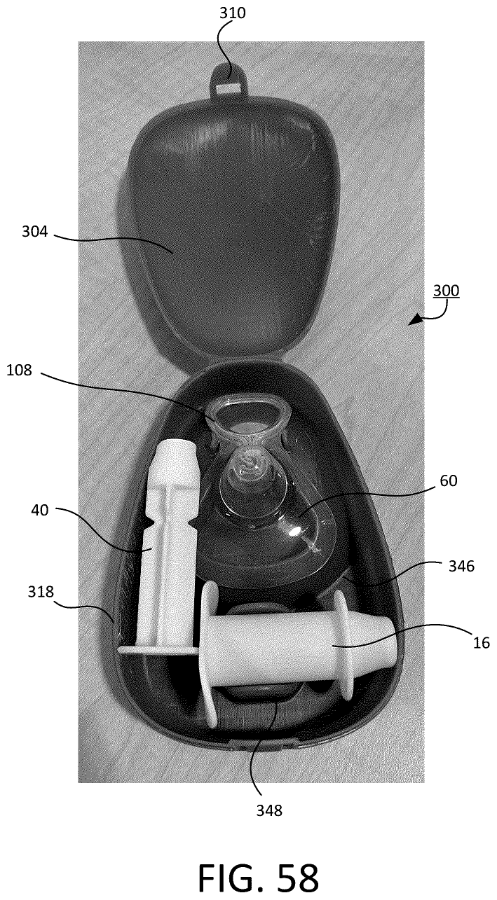

In step S6, dismantle the flush syringe 10 into two parts, the plunger 40 and the barrel 16 and arrange the two parts into the sterilizing storage case by placing the barrel 16 into a parabolic projection and placing the plunger 40 in a vertical orientation such that the barrel 16 is adjacent to the tip base 32 as shown in FIG. 58. Proceed to step S7.

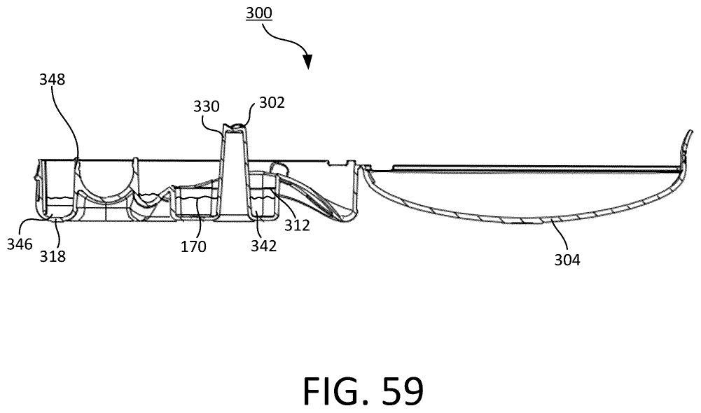

In step S7, overfill the receptacle reservoir with the cleaning liquid passing the fill line indicator such that the cleaning liquid fills into the receptacle basin until the cleaning liquid reaches the base of the parabolic projection, as shown in FIG. 59. Proceed to step S4 and S5 respectively.

As shown in FIG. 57, the generated steam then tracks upward from the receptacle reservoir passing the receptacle projection and then enters the nipple shield 60 through the plurality of notches and into the fluid channel, then through the translucent flow indicator and out from the plurality of openings, whereby the nipple shield 60 is rinsed and sterilized by the generated steam. For example, steam may push any residue left behind within the fluid channel, such as breast milk, from the nipple shield 60. The combination of the heat and possible sterilizing capability of the cleaning liquid sterilize the nipple shield for continued safe usage. In alternative embodiments where the sterilizing storage case has the at least one channel on the receptacle projection, or the at least one internal channel within the receptacle projection, the steam may be guided by the at least one channel or the at least one internal channel upward from the receptacle reservoir and into the fluid channel. It is to be understood that the sterilizing storage case may have any combination of the plurality of notches, the at least one channel and/or the at least one internal channel, suitably capable of directing the flow of steam from the receptacle reservoir upward into the nipple shield 60 or equivalent mating device. Further, the steam generated is sterilizes the exterior of the plunger 40 and barrel 16, and my also enter the fluid reservoir 22 effectively sterilizing the same.

The sterilizing storage case may also be placed into a container having hot water or equivalent liquid such that the cleaning liquid, contained within the closed sterilizing case and nipple shield housed within, may convert the cleaning liquid into steam as described by the present subject disclosure. For example, the sterilizing storage case may be placed into a pot of boiling water capable of raising the temperature of the cleaning liquid to generate the steam. Furthermore, it is to be understood that in alternative embodiments the material characteristics of the sterilizing storage case may include, but are not limited to: metal, plastic, synthetics or any other material suitably capable of withstanding the temperature needed raise the temperature of the cleaning liquid to generate the steam as described in this subject disclosure.

It is to be understood that the sterilizing storage case may be used to sterilize other infant related products such as but limited to: pacifiers, bottle nipples, teething toys or any other infant products that may have crevices that require rinsing, cleaning and/or sterilizing. Furthermore, it is to be understood that the sterilizing storage case 300 may be used to store and safe keep said related items, such as a nipple shield, for traveling purposes or to keep exposure to the atmosphere limited.

The material characteristics of the sterilizing storage case 300 may include, but are not limited to: plastic, polymers, or other suitable materials capable of withstanding the heating conditions required to generate steam as described in the subject matter of this disclosure.

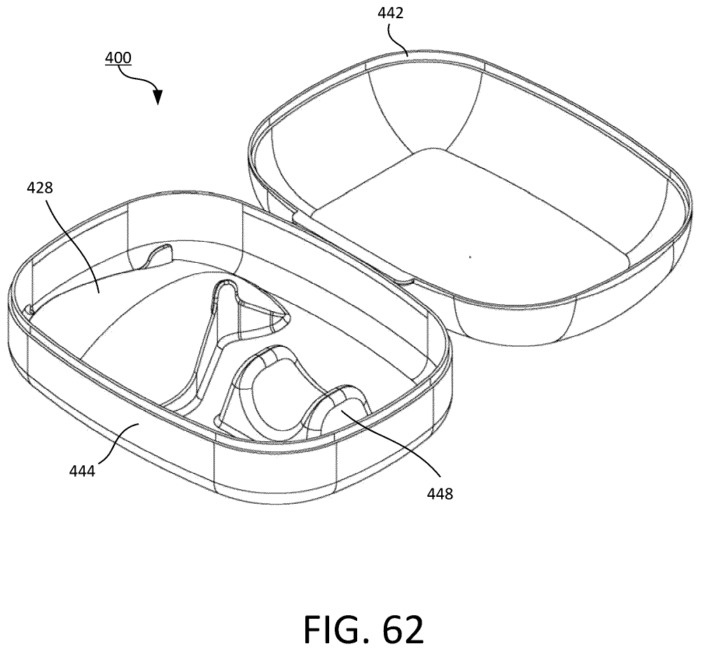

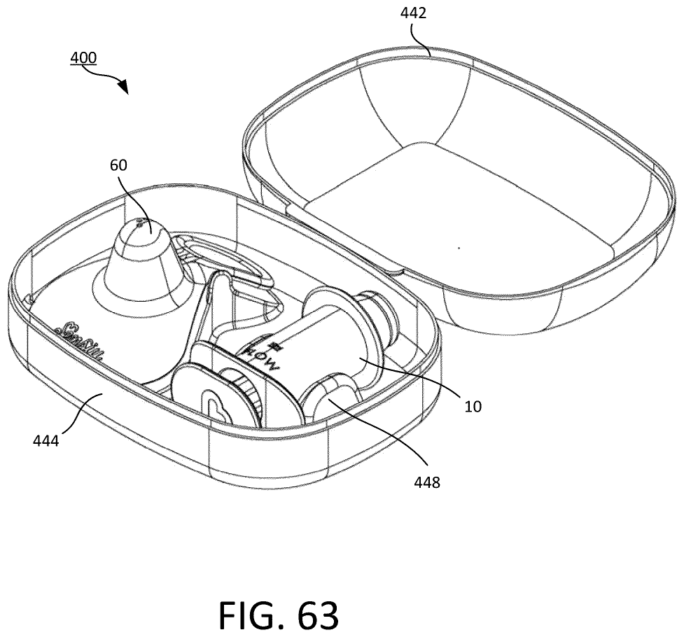

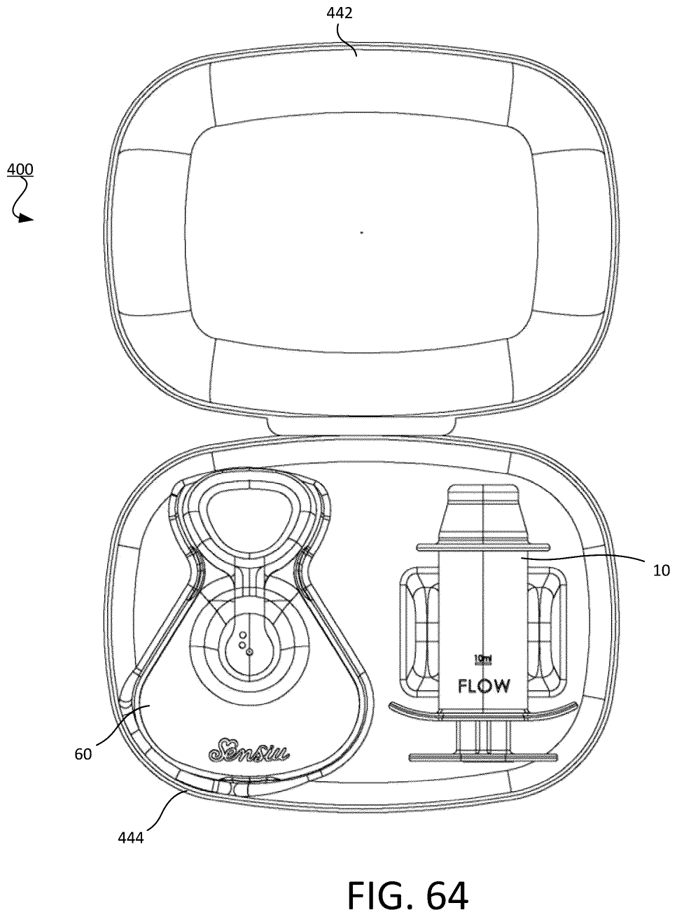

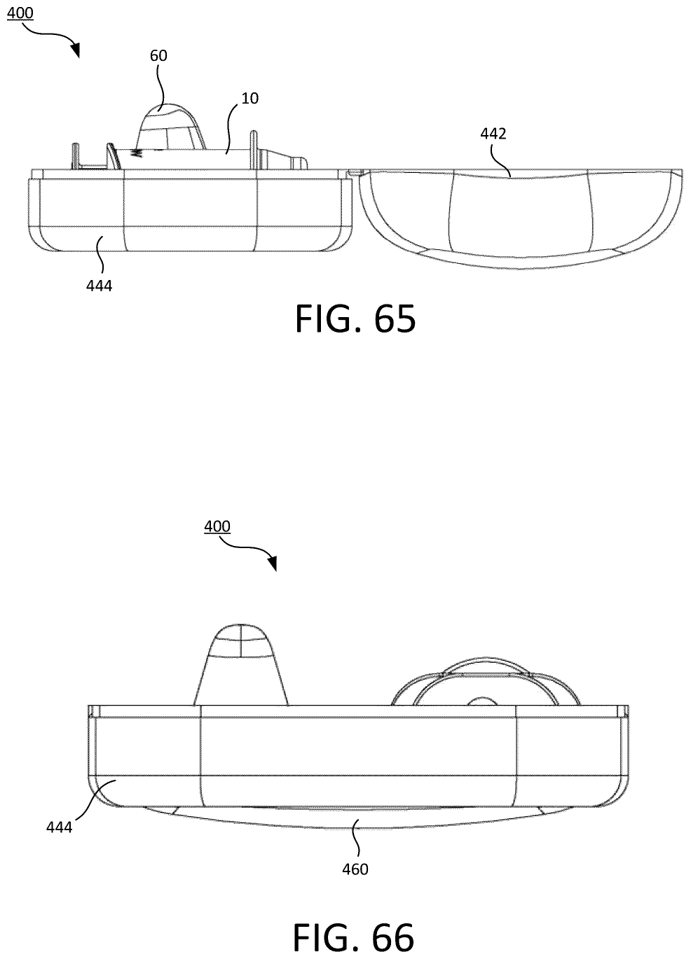

FIGS. 61-66, depicts another embodiment of a sterilizing storage case 400 that may have alternative embodiments that are capable of performing the features as described in the present subject disclosure. As shown, the sterilizing storage case 400 may take on a variety of different shapes and constructions. The sterilizing storage case 400 is more rectangular shaped having an upper curved case cover 442, and a receptacle reservoir 444 with a substantially flat bottom. An arched support 428 and a parabolic projection are enclosed by the receptacle reservoir 444 to align and secure the nipple shield 60 and flush syringe 10 respectively, during storage and cleaning.

As shown in FIG. 66 the receptacle reservoir 444 may be configured to include a heating plate 460 that is capable of generating steam out of the cleaning liquid 170. The heating plate 460 may include an internal power supply (not shown) such as, but not limited to, a rechargeable battery. The internal power supply may connected to a heating component (not shown) to raise the temperature of the heating plate 460 to generate steam out of the cleaning liquid 170. The heating component may be, but is not limited to, heating coils. The benefits of including the heating plate 460 would permit the user portability when traveling to safe keep the nipple shield 60 or other products within the sterilizing storage case 400, while also having the ability to sterilize the products.

The illustrations and examples provided herein are for explanatory purposes and are not intended to limit the scope of the appended claims. It will be recognized by those skilled in the art that changes, or modifications may be made to the above-described embodiments without departing from the broad inventive concepts of the invention. It is understood therefore that the invention is not limited to the embodiments which are described but is intended to cover all modifications and changes within the scope and spirit of the subject disclosure.

The foregoing disclosure of the exemplary embodiments of the present subject disclosure has been presented for purposes of illustration and description. It is not intended to be exhaustive or to limit the subject disclosure to the precise forms disclosed. Many variations and modifications of the embodiments described herein will be apparent to one of ordinary skill in the art in light of the above disclosure. The scope of the subject disclosure is to be defined only by the claims appended hereto, and by their equivalents.

Further, in describing representative embodiments of the present subject disclosure, the specification may have presented the method and/or process of the present subject disclosure as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present subject disclosure should not be limited to the performance of their steps in the order written, and one skilled in the art can readily appreciate that the sequences may be varied and still remain within the spirit and scope of the present subject disclosure.

What is claimed is:

1. A flush syringe, comprising:
a cylindrical container with an open first end and a substantially hollow tip with an opening at a second end, having a flat surface at the second end that expands radially outward into a rounded lip outer circumference that then extends into an annular wall that expands radially downward and outward to a base; and
a splash guard extending radially outward from the annular wall located between the open first end of the cylindrical container and the tip,
wherein the cylindrical container, the tip, and the splash guard all have a continuous uniform wall of equal thickness, and wherein an interior cavity is defined and bounded by the hollow tip and the cylindrical container to contain a fluid reservoir; and
a plunger having a stopper that is secured to a post, wherein the plunger is inserted into the first open end and the stopper is configured to sealingly engage with an interior portion of the hollow tip to forcibly dispense a fluid stored within the fluid reservoir through the outlet.

2. The flush syringe of claim 1, wherein the rounded lip outer circumference extends inward before extending into the annular wall; and wherein the stopper is removably secured to the post.

3. The flush syringe of claim 2, wherein an outer surface of the stopper has a rounded outer edge that is configured to mate with a complementary recess on the interior portion of the hollow tip.

4. The flush syringe of claim 1, wherein an outer surface of the stopper includes one or more annular bulges which provide a friction fit with an inner surface on the interior portion of the hollow tip.

5. The flush syringe of claim 1, wherein an outer surface of the stopper is shaped to be substantially parallel with the annular wall that expands radially downward and outward on the outer surface of the cylindrical container.

6. The flush syringe of claim 1, wherein the plunger further includes a full line indicator which indicates a maximum amount of the fluid of the fluid reservoir.

7. A sterilizing storage case, comprising:
a receptacle having a perimeter wall surrounding a base,
wherein the base further comprises:
a reservoir container adapted to hold a fluid;
a projection extending upward from within the reservoir container, the projection having a recessed wall surrounded by at least one opening within a tip of the projection through which the fluid traverses into a center portion of the projection; and
a cover connected to the receptacle adapted to close the receptacle.

8. The sterilizing storage case of claim 7, wherein the recessed wall is surrounded by an outer wall having at least one solid portion and at least one opening at the tip of the projection through which steam vents through.

9. The sterilizing storage case of claim 7, wherein the recessed wall is surrounded by an outer wall having a plurality of solid portions and openings disposed at the tip of the projection.

10. The sterilizing storage case of claim 9, wherein the fluid traverses through the openings.

11. The sterilizing storage case of claim 7, wherein the at least one channel fluidly communicates within the projection at a lower end adjacent to the reservoir container.

12. The sterilizing storage case of claim 7, further comprising a holder.

13. The sterilizing storage case of claim 12, wherein the projection is adapted to secure a nipple shield, and the holder is adapted to secure a syringe.

14. The sterilizing storage case of claim 7, further comprising apertures through which a steam may vent through.

15. The sterilizing storage case of claim 7, further comprising apertures disposed in the sterilizing storage case through which fluid travels therethrough.

16. A method of cleaning contents within a sterilizing storage case, comprising:
providing the sterilizing storage case having a projection extending upward with a recessed base and a crenellated tip and having at least one opening disposed in the crenellated tip;
opening a cover of the sterilizing storage case;
filling a reservoir with a cleaning fluid;
placing the contents within the sterilizing storage case, and closing the cover; and
heating the cleaning fluid to a boiling temperature to produce steam.

17. The method recited in claim 16, wherein the contents include a nipple shield.

18. The method recited in claim 16, wherein the contents include a syringe.

19. The method recited in claim 16, wherein the contents include a nipple shield and syringe.

20. The method recited in claim 19, wherein a flow of the steam is adapted to travel through the at least one opening in the crenellated tip that is directed to a specific area of the nipple shield.
